(19) United States
(12) Patent Application Publication
(10) Pub. No.: US 2026/0276660 A1
(43) Pub. Date: Sep. 17, 2026

(54) DETERMINATION OF PROGESTOGENS NON-INVASIVELY IN ANIMALS

(71) Applicant: Vetcare Oy, Salo (FI)

(72) Inventors: Sari VIITALA, KUOPIO (FI); Tia PENNANEN, KUOPIO (FI)

(21) Appl. No.: 19/165,088

(22) PCT Filed: Mar. 14, 2024
(86) PCT No.: PCT/FI2024/050113

§ 371 Date: Sep. 12, 2025

(30) Foreign Application Priority Data
Mar. 15, 2023 (FI) 20235301

(51) Int. Cl. G01N 33/74

(52) U.S. Cl. CPC G01N 33/743; G01N 2333/575; G01N 2800/36

(57) ABSTRACT

The present disclosure relates to 5α-pregnane-3,6,20-triol isomer or 5α-pregnane-3,6,20-triol in a glucuronidated form in determination of progestogen or progestogens in a sample from a non-human animal. The present disclosure also relates to an in vitro method of determining progestogen or progestogens in a sample from a non-human animal.

### FIELD OF THE DISCLOSURE

The present disclosure relates to a compound, which is 5α-pregnane-3,6,20-triol isomer or 5α-pregnane-3,6,20-triol isomer in a glucuronidated form. The present disclosure also relates to use of 5α-pregnane-3,6,20-triol isomer or 5α-pregnane-3,6,20-triol isomer in a glucuronidated form in determination of progestogen or progestogens in an in vitro urine sample from a non-human animal. The present disclosure also relates to an in vitro method of determining progestogen or progestogens in a urine sample from a non-human animal.

### BACKGROUND

The dog's optimal breeding time is determined primarily by progesterone analysis of serum samples. In the analysis, an increase in the concentration of the serum progesterone hormone to a certain level indicates that ovulations occurred, as a result of which conception is possible.

During proestrus, which lasts usually 3 to 21 days, external female genital organs and perineum distend in a bitch. Vaginal discharge, which might be creamy or sanguinary, become usually visible (Concannon, 2011). The length and volume of the vaginal discharge is highly varying between individuals, and in some bitches secretes can be detected from proestrus to early diestrus.

The onset of behavioural oestrus is determined as the first day of the acceptance of coitus. If the dog is allowed to reproduce by itself, bitch is usually mated during the fertile timing. However, fertilization might demand copulations during multiple consecutive days. In practice and in research, this is usually laborious or even impossible to organize. In addition, as the mating behaviour is highly individual and sometimes hard to recognize, the monitoring of oestrus behaviour is not a specific tool for predicting the timing of fertile timing.

In canids, the stage of the oestrus can be estimated by vaginal cytology. During proestrus vaginal epithelial cells start to divide and is followed by morphological changes and keratinization in different oestrus phases. However, without any additional methods cytological analysis is not always applicable for determination of the optimal breeding time (Moxon et al., 2010). In addition, the vaginal secretes crystallize right after the peripheral estrogen concentration reaches the maximal concentration. The crystallization detection can be used with cytological analysis but is not accurate by itself for oestrus phase monitoring (England and Allen, 1989).

Measurement of the electrical resistance of the vaginal secretion is a common method to detect the optimal breeding and/or insemination time in farmed foxes. The electrical conductivity of the vaginal secretes changes when the electrical resistance increases and reaches the maximum level soon after the LH peak, which is considered as the first day of the oestrus. In dogs, the method is usable but not that common due to a slower and less significant resistance increase (Günzel et al., 1986).

The size and shape of ovaries changes due to the growing follicles in proestrus and oestrus, and blood flow increase in ovaries during the LH peak. These changes can be detected by ultrasonography. Ultrasonography provides the most accurate method for ovulation detection but is a demanding and time-consuming method. The distinguishing features in follicle size increase, preovulatory luteinization and ovulation are very delicate and demand experience in interpretation of ultrasonographic pictures. In addition, locating the ovaries, especially in large breeds, can be difficult or even impossible if the dog is not comfortable during the procedure. Professionals can detect on-going ovulations in 50-92% of cases, but it demands daily observations, or even multiple observations per day (Fontbonne and Malandain, 2006).

The first day of oestrus (Day 0) is often determined as the day when luteinizing hormone (lutropin, LH) reaches its peak value, called LH peak. Like LH, follicle stimulating hormone (follitropin, FSH) peak can be detected at the beginning of oestrus. These hormones are gonadotropins, which regulate gonadal functions such ovulation. At the end of the anestrus phase, FSH level increases and stimulates follicles to grow in size and numbers. In proestrus, the FSH level decreases and suddenly peaks. Luteinizing hormone level increases at the end of the anestrus phase, which stimulates the maturation of the follicles. LH peaks at the same time as the FSH, but the length of the peak is only a third of the FSH peak. The length of the LH peak is approximately 36 hours. The peaks of gonadotropins cause the ovulation in one to two days (Concannon, 2009). Due to the pulsatile excretion of gonadotropins, the detection of peak values demands daily blood samples or even multiple samples per day. However, determination of the LH peak is often considered as the most accurate method to predict the timing of ovulation. Besides enzyme immunoassays, LH can be detected from serum samples by rapid test (WITNESS LH Rapid test).

Follicles and corpora lutea secrete estrogens (estrone (E1), estradiol (E2), and estriol (E3)). In proestrus, maturating follicles produce estrogens, which decreases the FSH level (Concannon, 2009). Increasing estrogen level induces the external signs of oestrus and pheromone excretion. After reaching its maximum value, estrogen concentration suddenly decreases. Gonadotropin peaks in a few days after the estrogen level decreases. Estrogen measurements are usually performed by enzyme immunoassays from blood samples, but the utility of the assay in determination of the optimal breeding time is questionable: the estrogen levels vary significantly not only between individuals but also in a single animal between oestrus cycles (Frank et al., 2010).

Progesterone is produced in follicular theca and granulosa cells, corpora lutea and in adrenal cortex. In dogs, follicles start to luteinize right after the maximum capacity of the estrogen secretion is reached, already before LH peak. The preovulatory luteinization can be detected by increased serum progesterone level (~0.35→~0.8 ng/ml). During the LH peak the progesterone concentration is 1-2 ng/ml, and the excretion accelerates right after. During the ovulation, approximately two days after LH peak, progesterone level increases to ~6 ng/ml concentration. The secretion of progesterone increases in the early diestrus until the maximum concentration (14-80 ng/ml) is reached approximately in ten days or at the latest in three to five weeks after the LH peak. The excretion rate of progesterone is similar in non-pregnant and pregnant bitches. However, in pregnant bitches a sudden decrease of progesterone to basal level is detected usually 24 hours before parturition, as in non-pregnant bitches the decrease of progesterone is slower and may take even longer period of time (Concannon et al., 2009). Due to the preovulatory luteinization, serum progesterone analysis has been the most used method to detect optimal breeding time in bitches. However, drawing blood is invasive and unpleasant method to the dog. In addition, it is often required to collect blood samples several times, which causes significant expenses when blood is collected and analyzed by professionals.

Despite the advances in the technology to monitor oestrus cycle in an animal there remains a need for improved non-invasive methods and products.

### SUMMARY OF THE DISCLOSURE

An object of the present disclosure is to provide compounds, as well as uses, and methods thereof which overcome the above problems related to the presently used methods.

The present disclosure relates to a compound which comprises 5α-pregnane-3,6,20-triol isomer or 5α-pregnane-3,6,20-triol isomer in a glucuronidated form.

The present disclosure also relates to a use of 5α-pregnane-3,6,20-triol isomer or 5α-pregnane-3,6,20-triol isomer in a glucuronidated form in determination of progestogen or progestogens in an in vitro sample from a non-human animal, wherein the compound comprises 5α-pregnane-3,6,20-triol isomer or 5α-pregnane-3,6,20-triol isomer in a glucuronidated form.

The present disclosure also relates to an in vitro method of determining progestogen or progestogens in a sample from a non-human animal, wherein the method comprises the steps of providing an in vitro urine sample from a non-human animal, measuring an amount of 5α-pregnane-3,6,20-triol isomer or 5α-pregnane-3,6,20-triol glucuronide isomer in said sample, determining the presence of luteinized follicular theca and/or granulosa cells based on the amount of 5α-pregnane-3,6,20-triol isomer or 5α-pregnane-3,6,20-triol glucuronide isomer in the sample, wherein the progestogen level in the sample of the non-human animal is indicative by the presence of luteinized follicular theca and/or granulosa cells.

Specifically, the disclosure concerns 5α-pregnane-3,6,20-triol isomer or 5α-pregnane-3,6,20-triol glucuronide isomer and their use to determine the oestrus cycle phase in an animal, such as a canid.

### BRIEF DESCRIPTION OF THE FIGURES

FIG. 1 illustrates extracted ion chromatograms (EIC, m/z 495) from LC-MS data of commercially available PdG (pregnanediol-3-glucuronide, 5β-pregnane-3α,20α-diol glucuronide), enzymatically glucuronidated 5β-pregnane-3α,20α/β-diols and two diestrus phase urine samples. A) commercially available PdG, B) enzymatically glucuronidated 5β-pregnane-3α,20α-diol, C) enzymatically glucuronidated 5β-pregnane-3α,20β-diol, D) diestrus phase urine sample from bitch A, and E) diestrus phase urine sample from bitch B. The retention time of the PdG is marked by a solid box and retention time of the 20β-form of PdG is marked by a dotted line box. In dogs, the 20β-form of PdG has a higher concentration than the 20α-form.

FIG. 2 illustrates comparison of LC-MS data between urinary PdGα (20α-form of PdG) and PdGβ (20β-form of PdG) levels in eight bitches (EIC area m/z 495 average±SD). The β-reduction is favored at the carbonyl group in C20 of PdG.

FIG. 3 illustrates extracted ion chromatograms (EIC, m/z 511) from LC-MS data of enzymatically glucuronidated 5β-pregnane-3α,17α,20α/β-triols and two diestrus phase urine samples. A) enzymatically glucuronidated 5β-pregnane-3α,17α,20α-triol (17α-PtGα, 20α-form), B) enzymatically glucuronidated 5β-pregnane-3α,17α,20β-triol (17α-PtGβ, 20β-form), C) diestrus phase urine sample from bitch A, D) diestrus phase urine sample from bitch B, E) zoomed EIC (bitch A), and F) zoomed EIC (bitch B). The retention time of 17α-PtGα is marked by a solid box and retention time of 17α-PtGβ is marked by a dotted line box. In dogs, 20β-reduction is more common than 20α-reduction at the C20 carbonyl group of 17α-PtG, as in the PdG. In dogs, 17α-PtGα/P is excreted in urine only in small amounts, whereas the more abundant metabolite with the same m/z 511 can be seen at retention time of 6.4 minutes.

FIG. 4 illustrates profiles of urinary 5β-pregnane-3α,17α,20α/β-triol-glucuronide (average area±SD from extracted ion chromatogram m/z 511 at retention time of 8.7 minutes) with serum progesterone (P4, a-b; ng/ml±SD) and serum 17α-hydroxyprogesterone (17α-OHP, c-d; pg/ml±SD) in eight bitches measured by LC-MS. Day 0 was considered as the day when serum estradiol (E2) reached its maximum concentration. In the figure, the 17α-PtGα means 5β-pregnane-3α,17α,20α-triol glucuronide (20α-form) and the 17α-PtGβ means 5β-pregnane-3α,17α,20β-triol glucuronide (20β-form). As the 20α- and 20β-forms of 17α-PtGs are close-eluting, the area is calculated by integrating the split double peak manually.

FIG. 5 illustrates comparison between serum progesterone (average P4 ng/ml±SD) and urinary, unidentified pregnanetriol glucuronide (PtGX, retention time 6.4 min, average peak area/Cr±SD) in eight bitches, measured by LC-MS. The profile of the PtGX follows the serum P4 profile.

FIG. 6 illustrates LC-MS data about two close-eluting isomers of PtG (pregnanetriol glucuronide) in the urine of two bitches (A and B). The first eluted PtG (PtG1) is the most abundant urinary PtG in bitch A and its retention time is marked as dashed line. The second eluted PtG (PtG2) is the most abundant urinary PtG in bitch B and is marked as dotted line.

FIG. 7 illustrates extracted ions corresponding to pregnanediol glucuronides (PdGs) in LC-MS data (EIC m/z 495). The retention time of PdG (5β-pregnane-3α,20α-diol-3-glucuronide), the major metabolite for progesterone in human, is marked as dashed line (retention time 8.7 min). In a dog, the PdG is found only in traces.

FIG. 8 illustrates extracted ions corresponding to pregnanetriol glucuronides (PtGs) in LC-MS data (EIC m/z 511). The retention time of major metabolite for 17α-hydroxyprogesterone in human, 5β-pregnane-3α,17α-20α-triol glucuronide, is marked as dashed line (retention time 8.46 min). In a dog, the 17α-PtG is excreted as 5β-pregnane-3α,17α,20β-triol glucuronide (20β-form of 17α-PtG), with a retention time only 0.05 minutes later (retention time 8.51 min). The most abundant pregnanetriol glucuronide in a dog is found at retention time of 6.38 minutes.

FIG. 9 illustrates extracted ions corresponding to pregnanediol-one glucuronides in LC-MS data (EIC m/z 509). Only a few peaks (marked as dashed lines) are found at same retention time in a dog and human sample.

FIG. 10 illustrates extracted ions corresponding to pregnane-ol-dione glucuronides in LC-MS data (EIC m/z 507). In a dog, only one peak can be found (marked as dashed line). The same peak is found in a human sample.

FIG. 11 illustrates the production of hydroxyprogesterones (OHPs) from progesterone by ovarian and hepatic enzymes in the S9 fraction based on LC-MS data. The lines A-D illustrate the retention times of reference hydroxyprogesterones: A) 6α-OHP, B) 16α-OHP, C) 6β-OHP, and D) 17α-OHP. Ovarian S9 enzymes have high 17α-hydroxylase activity. Hepatic S9 enzymes have high 6α-, 6β- and 16α-hydroxylase activity.

FIG. 12 illustrates extracted ion chromatogram from LC-MS data of pregnanetriol glucuronides (PtGs, m/z 511) formed by reduction and glucuronidation of 16α-hydroxyprogesterone (A), and two diestrus phase urine samples from two bitches (B and C). The PtG1 is marked as dashed line and the PtG2 as dotted line. The most abundant synthetic PtGs are marked as solid lines.

FIG. 13 illustrates extracted ion chromatogram from LC-MS data of pregnanetriol glucuronides (PtGs, m/z 511) formed by reduction and glucuronidation of 6p-hydroxyprogesterone (A), and two diestrus phase urine samples from two bitches (B and C). The PtG1 is marked as dashed line and the PtG2 as dotted line. The most abundant synthetic PtGs are marked as solid lines.

FIG. 14 illustrates extracted ion chromatogram from LC-MS data of pregnanetriol glucuronides (PtGs, m/z 511), in which A) illustrates reference steroid formed by reduction and glucuronidation of 6α-hydroxyprogesterone, B) diestrus urine sample, C) diestrus urine sample spiked with 0.5 μM reference steroid, and D) diestrus urine sample spiked with 1.5 μM reference steroid. The PtG1 is marked as dashed line and the PtG2 as dotted line. The most abundant synthetic PtG is marked as solid lines.

FIG. 15 illustrates extracted ion chromatogram from LC-MS data of pregnanediol-ones (m/z 509), in which A and B illustrates m/z 509 ions in two diestrus phase urine samples in two bitches and C illustrates the reference steroid of glucuronidated 5β-pregnane-3α,6α-diol-20-one. Reference glucuronides are marked as solid lines, and endogenous pregnanediol-ones are marked with dash dot lines.

FIG. 16 illustrates extracted ion chromatogram from LC-MS data of pregnanetriol glucuronides (PtGs, m/z 511) formed by reduction and glucuronidation of 5β-pregnane-3α,6α-diol-20-one (A), and two diestrus phase urine samples from two bitches (B and C). The PtG1 is marked as dashed line and the PtG2 as dotted line. The most abundant synthetic PtGs are marked as solid lines.

FIG. 17 illustrates extracted ion chromatogram from LC-MS data of pregnanetriol glucuronides (PtGs, m/z 511) formed by A) deacetylation and glucuronidation of 5β-pregnane-3α,6α,20α-triol-20-acetate, B) deacetylation and glucuronidation of 5β-pregnane-3α,6α,20β-triol-20-acetate, C) and D) two diestrus phase urine samples from two bitches. The PtG1 is marked as dashed line and the PtG2 as dotted line. The synthetic PtGs are marked as solid lines.

FIG. 18 illustrates extracted ion chromatogram from LC-MS data of pregnanetriol glucuronides (PtGs, m/z 511) formed by A) reduction and glucuronidation of 5α-pregnane-3β,6α-diol-20-one, B) and C) two diestrus phase urine samples from two bitches. The most abundant synthetic PtGs are marked as solid lines, excluding the one with same retention time with the PtG1, which is marked as dashed line. The PtG2 is marked as dotted line.

FIG. 19 illustrates extracted ion chromatogram from LC-MS data of pregnanetriol glucuronides (PtGs, m/z 511) formed by A) reduction and glucuronidation of 5α-pregnane-3β,20β-diol-6-one, B) and C) two diestrus phase urine samples from two bitches. The most abundant synthetic PtGs are marked as solid lines, excluding the one with same retention time with the PtG1, which is marked as dashed line. The PtG2 is marked as dotted line.

FIG. 20 illustrates extracted ion chromatogram from LC-MS data of pregnanetriol glucuronides (PtGs, m/z 511) formed by A) reduction and glucuronidation of pregnenolone, B) and C) two diestrus phase urine samples from two bitches. The reference steroid was extracted by solid-phase extraction (SPE) and eluted in 50% methanol.

FIG. 21 illustrates extracted ion chromatogram from LC-MS data of pregnanetriol glucuronides (PtGs, m/z 511) formed by A) glucuronidation of 5α-pregnane-3β,6α,20α-triol (solid lines), B) glucuronidation of 5α-pregnane-3β,6α,20β-triol (dashed line), C) and D) two diestrus phase urine samples from two bitches. The PtG1 is marked as dashed line and the PtG2 as dotted line.

FIG. 22 illustrates extracted ion chromatogram from LC-MS data of pregnanetriol glucuronides (PtGs, m/z 511) formed by A) synthetic 5α-PtG (5α-pregnane-3,6,20-triol glucuronide), B) diestrus phase urine sample spiked with 1 μM 5α-PtG, C) diestrus phase urine sample spiked with 0.5 μM 5α-PtG, D) diestrus phase urine sample without addition of the 5α-PtG. The PtG1 is marked as dashed line and the PtG2 as dotted line. The increase in the area of the urinary PtG1 peak was linear with the addition of the 5α-PtG.

FIG. 23 illustrates extracted ion chromatogram from LC-MS data of pregnanetriol glucuronides (m/z 511) formed by A) 5α-PtG (5α-pregnane-3,6,20-triol glucuronide), B) diestrus phase urine sample spiked with 1 μM 5α-PtG, C) diestrus phase urine sample spiked with 0.5 μM 5α-PtG, D) diestrus phase urine sample without addition of the 5α-PtG. The PtG1 is marked as dashed line and the PtG2 as dotted line. The increase in the area of the urinary PtG1 peak was linear with the addition of the reference. The urine sample was from another individual than in FIG. 20.

FIG. 24 illustrates urinary 5α-PtG concentration (sum of PtG1 and PtG2; nM±SEM) and serum progesterone concentration (ng/ml±SEM) in eight bitches measured by LC-MS. Urinary PtG concentration follows the serum progesterone concentration. A) serum and urinary concentrations are measured as same day results, B) urine results are considered as a result of progesterone from a previous day. Ovulation was estimated to occur when serum progesterone was 5-6 ng/ml or two days after 2 ng/ml of progesterone (estimated LH peak).

FIG. 25 illustrates the average (±SEM) peak areas of urinary 5α-PtG (area of co-eluting peaks PtG1 and PtG2) and urinary PdGβ (20β-form of the PdG) in eight bitches. Peaks were integrated from extracted ion chromatograms from LC-MS data. A) Average areas displayed in same axis of area, B) average areas are displayed in separate axis; the PdGβ resemble the profile of the 5α-PtG. Ovulation was estimated to occur when serum progesterone was 5-6 ng/ml or two days after 2 ng/ml of progesterone (estimated LH peak).

FIG. 26 illustrates the average concentration (nM±SEM) of urinary 5α-PtG from a series of 60 urine samples collected from 52 bitches as measured by EIA.

FIG. 27 illustrates the average concentration (nM±SEM) of urinary 5α-PtG and serum concentration (ng/ml±SEM) of progesterone in 27 bitches. LH peak was estimated to occur when serum progesterone was approximately 2 ng/ml or two days before assumed ovulation (serum progesterone was 5-6 ng/ml).

FIG. 28 illustrates a lateral flow immunochromatographic test strip, which includes sample pad, conjugate pad, nitrocellulose membrane and absorbent pad.

### DETAILED DESCRIPTION

As described above it is important to determine the stage of oestrus to detect the optimal breeding and/or insemination time in animals.

It would therefore be beneficial to provide a non-invasive, cost-effective, and quick method for monitoring the oestrus cycle.

Thus, it is provided in the present disclosure a novel method to monitor oestrus cycle in an animal, especially in a non-human animal, for example a canine. Monitoring oestrus cycle from excrete samples, such as urine, saliva and/or fecal samples, is painless and cost-effective. Preferably the sample is a urine sample. Rapid tests, such as urine rapid tests, provide inexpensive tools for animal breeders, for example for dog owners and breeders to detect the optimal breeding time at home without stressful procedures such as blood or vaginal smear sampling. Sample can be collected with common houseware at no extra costs.

Serum progesterone analysis is a commonly used method to determine the optimal breeding time in a dog. It can be used in any oestrus cycle phase to estimate the oestrus cycle phase in a bitch. However, the metabolism of progestogens in canids is unknown, why existing non-invasive methods to measure excreted steroid metabolites are not usable in canids.

The present inventors noticed that in canids, the progesterone (pregn-4-ene-3,20-dione) hormone metabolism is unique among mammals. Whereas the progestogens are usually reduced as 50-pregnane structured metabolites as 5β-pregnane-3,20-diol glucuronides (PdG) and 5β-pregnane-3,17,20-triol glucuronides (17α-PtG) in mammals (Niemuller et al., 1993; O'Connor et al., 2003), they are reduced as 5α-pregnane derivatives in a canid, such as dog. In human, increased amount of 5α-reduced metabolites can be a sign of various diseases such as breast cancer or intrahepatic cholestasis of pregnancy (Meng et al., 1997; Wiebe, 2005).

In dogs, only traces of the PdG are excreted to urine. In the other hand, the concentration of 5β-pregnane-3α,17α,20α-triol glucuronides (17α-PtGs) (both 20α and 20β isomers) fluctuates through the proestrus and oestrus phase. The urinary pregnane-3,17,20-triol glucuronides do not follow the serum progesterone or 17α-hydroxyprogesterone and are not usable biomarkers for progestogens. Interestingly, in dogs the 20β-reduction of progesterone metabolites is far more common than 20α-reduction: in dog urine the corresponding metabolites to the PdG and the 17α-PtG are 5β-pregnane-3α,20β-diol and 5β-pregnane-3α,17α,20β-triol glucuronides.

Instead of 17α-hydroxylation, the progestogens are excreted mostly as pregnane-3,6,20-triol glucuronide isomers in dogs. In human, 5α-pregnane-3,6,20-triols are biomarkers for cancers (Fennessey et al., 1986; Suzuki et al., 2002). Results indicate that due to unique metabolism in dogs, the existing oestrus/menstrual cycle monitoring or progestogen measuring methods used for human or other mammals, are not usable to canids. These 5α-reduced pregnane-3,6,20-triols presented in the disclosure and glucuronidated forms of them have never been used as biomarkers of progestogens in oestrus monitoring before. The present disclosure provides new tools for non-invasive progestogen detection in canids.

Especially, the present disclosure pertains different methods to determinate oestrus cycle phase in a bitch (Canis familiaris) by urinary measurements of progestogen metabolites. The disclosure provides methods to discover if dog ovaries are secreting progestogens due to preovulatory luteinization or luteinized follicles. Methods can be used to detect the optimal breeding time (mating or inseminating) in a bitch, and the methods are easily applicable to other canids too.

As blood circulatory steroid hormones are excreted in the urine, saliva and feces as metabolites, the concentration of the metabolites can be determined by a suitable method known by a skilled person in the art. The most common metabolites of progesterone and 17α-hydroxyprogesterone, PdG and 17α-PtG, are not useful biomarkers for monitoring dog oestrus.

The present disclosure relates to 5α-pregnane-3,6,20-triol or stereoisomers thereof.

The molecule of the present disclosure comprises a main structure (5α-pregnane-3,6,20-triol) and a side structure (glucuronide).

The molecular formula of the 5α-pregnane-3,6,20-triol is C₂₁H₃₆O₃ and the molecular weight of the 5α-pregnane-3,6,20-triol is 336.51 g/mol.

The molecular formula of the 5α-pregnane-3,6,20-triol glucuronide is C₂₇H₄₄O₉ and the molecular weight of the 5α-pregnane-3,6,20-triol glucuronide is 512.63 g/mol.

The general formula of the molecule is 5α-pregnane-3z,6z,20z-triol. The letter z represents the α or R configuration of the hydroxyl group of each chiral carbon. The C20 configuration can also be represented in R and S form.

The molecule can exist either as free form 5α-pregnane-3z,6z,20z-triol or as glucuronidated form 5α-pregnane-3z,6z,20z-triol glucuronide, in which one of its three hydroxyl groups is conjugated.

Due to both the chirality of the molecule and the alternative positions of the glucuronide, the molecule has several different possible structures. The formula (I) below reflects the structure of the molecule when the glucuronic acid is conjugated at the C3 position. Thus, the molecule of the formula (I) is 5α-pregnane-3z,6z,20z-triol-3-glucuronide. Hydroxylated chiral carbons are highlighted with dashed rectangles.

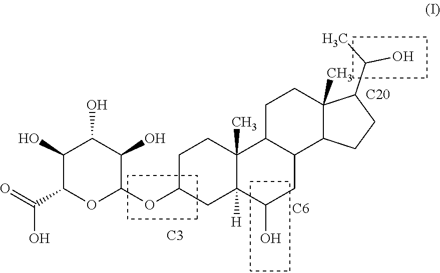

Chiral carbons in dashed rectangles (C3, C6 ja C20) can be in α or β configuration (C20 alternatively in S or R configuration). Glucuronic acid can be conjugated from C3, C6 and C20.

There are several alternative spellings for the molecule 5α-pregnane-3z,6z,20z-triol and its C3 glucuronide form, below are some examples of the synonyms.

Pregnane-3,6,20-triol; 5α-pregnane-3,6,20-triol; pregnane-3,6,20-triol glucuronide; pregnane-3,6,20-triol-3-glucuronide; 5α-pregnane-3,6,20-triol glucuronide; 5α-pregnane-3,6,20-triol-3-glucuronide; 5α-pregnane-3z,6z,20z-triol; 5α-pregnane-3z,6z,20z-triol-3-O-z-D-glucuronide; (3z,5alpha,6z,20z)-3,6,20-trihydroxypregnan; (3z,5alpha,6z,20z)-3,6,20-trihydroxypregnan-3-yl-z-D-glucopyranosiduronic acid; (3z,5 alpha,20z)-6,20-dihydroxypregnane; (3z,5 alpha,20z)-6,20-dihydroxypregnane-3-yl-z-D-glucopyranosiduronic acid; 6z,20z-dihydroxy-5α-pregnane; 6z,20z-dihydroxy-5α-pregnane-3z-yl-z-D-glucopyranosiduronic acid

There are eight stereoisomers for the molecule 5α-pregnane-3,6,20-triol and 48 stereoisomers for its glucuronide. All stereoisomers with one spelling are listed below in Tables 1 and 2.

TABLE 1
Stereoisomers existing for the molecule 5α-pregnane-3,6,20-triol.
5α-pregnane-3α,6α,20α-triol
5α-pregnane-3β,6α,20α-triol
5α-pregnane-3α,6β,20α-triol
5α-pregnane-3α,6α,20β-triol
5α-pregnane-3β,6β,20α-triol
5α-pregnane-3β,6α,20β-triol
5α-pregnane-3α,6β,20β-triol
5α-pregnane-3β,6β,20β-triol

TABLE 2
Stereoisomers existing for the molecule 5α-pregnane-3,6,20-triol glucuronide.

| | |
|---|---|
| 5α-pregnane-3α,6α,20α-triol-3-O-α-D- | 5α-pregnane-3α,6α,20α-triol-3-O-β-D- |
| glucuronide | glucuronide |
| 5α-pregnane-3β,6α,20α-triol-3-O-α-D- | 5α-pregnane-3β,6α,20α-triol-3-O-β-D- |
| glucuronide | glucuronide |
| 5α-pregnane-3α,6β,20α-triol-3-O-α-D- | 5α-pregnane-3α,6β,20α-triol-3-O-β-D- |
| glucuronide | glucuronide |
| 5α-pregnane-3α,6α,20β-triol-3-O-α-D- | 5α-pregnane-3α,6α,20β-triol-3-O-β-D- |
| glucuronide | glucuronide |
| 5α-pregnane-3β,6β,20α-triol-3-O-α-D- | 5α-pregnane-3β,6β,20α-triol-3-O-β-D- |
| glucuronide | glucuronide |
| 5α-pregnane-3β,6α,20β-triol-3-O-α-D- | 5α-pregnane-3β,6α,20β-triol-3-O-β-D- |
| glucuronide | glucuronide |
| 5α-pregnane-3α,6β,20β-triol-3-O-α-D- | 5α-pregnane-3α,6β,20β-triol-3-O-β-D- |
| glucuronide | glucuronide |
| 5α-pregnane-3β,6β,20β-triol-3-O-α-D- | 5α-pregnane-3β,6β,20β-triol-3-O-β-D- |
| glucuronide | glucuronide |
| 5α-pregnane-3α,6α,20α-triol-6-O-α-D- | 5α-pregnane-3α,6α,20α-triol-6-O-β-D- |
| glucuronide | glucuronide |
| 5α-pregnane-3β,6α,20α-triol-6-O-α-D- | 5α-pregnane-3β,6α,20α-triol-6-O-β-D- |
| glucuronide | glucuronide |
| 5α-pregnane-3α,6β,20α-triol-6-O-α-D- | 5α-pregnane-3α,6β,20α-triol-6-O-β-D- |
| glucuronide | glucuronide |
| 5α-pregnane-3α,6α,20β-triol-6-O-α-D- | 5α-pregnane-3α,6α,20β-triol-6-O-β-D- |
| glucuronide | glucuronide |
| 5α-pregnane-3β,6β,20α-triol-6-O-α-D- | 5α-pregnane-3β,6β,20α-triol-6-O-β-D- |
| glucuronide | glucuronide |
| 5α-pregnane-3β,6α,20β-triol-6-O-α-D- | 5α-pregnane-3β,6α,20β-triol-6-O-β-D- |
| glucuronide | glucuronide |
| 5α-pregnane-3α,6β,20β-triol-6-O-α-D- | 5α-pregnane-3α,6β,20β-triol-6-O-β-D- |
| glucuronide | glucuronide |
| 5α-pregnane-3β,6β,20β-triol-6-O-α-D- | 5α-pregnane-3β,6β,20β-triol-6-O-β-D- |
| glucuronide | glucuronide |
| 5α-pregnane-3α,6α,20α-triol-20-O-α-D- | 5α-pregnane-3α,6α,20α-triol-20-O-β-D- |
| glucuronide | glucuronide |
| 5α-pregnane-3β,6α,20α-triol-20-O-α-D- | 5α-pregnane-3β,6α,20α-triol-20-O-β-D- |
| glucuronide | glucuronide |
| 5α-pregnane-3α,6β,20α-triol-20-O-α-D- | 5α-pregnane-3α,6β,20α-triol-20-O-β-D- |
| glucuronide | glucuronide |
| 5α-pregnane-3α,6α,20β-triol-20-O-α-D- | 5α-pregnane-3α,6α,20β-triol-20-O-β-D- |
| glucuronide | glucuronide |
| 5α-pregnane-3β,6β,20α-triol-20-O-α-D- | 5α-pregnane-3β,6β,20α-triol-20-O-β-D- |
| glucuronide | glucuronide |
| 5α-pregnane-3β,6α,20β-triol-20-O-α-D- | 5α-pregnane-3β,6α,20β-triol-20-O-β-D- |
| glucuronide | glucuronide |
| 5α-pregnane-3α,6β,20β-triol-20-O-α-D- | 5α-pregnane-3α,6β,20β-triol-20-O-β-D- |
| glucuronide | glucuronide |
| 5α-pregnane-3β,6β,20β-triol-20-O-α-D- | 5α-pregnane-3β,6β,20β-triol-20-O-β-D- |
| glucuronide | glucuronide |

The present disclosure describes a compound which comprises 5α-pregnane-3,6,20-triol isomer or 5α-pregnane-3,6,20-triol isomer in a glucuronidated form.

In an embodiment the compound comprises 5α-pregnane-3,6,20-triol isomer.

In an embodiment the compound is selected from the group consisting of 5α-pregnane-3α,6α,20α-triol, 5α-pregnane-3β,6α,20α-triol, 5α-pregnane-3α,6β,20α-triol, 5α-pregnane-3β,6β,20α-triol, 5α-pregnane-3α,6α,20β-triol, 5α-pregnane-3α,6α,20β-triol, 5α-pregnane-3α,6α,20β-triol, and 5α-pregnane-3β,6β,20β-triol.

In an embodiment the compound comprises 5α-pregnane-3,6,20-triol glucuronide isomer.

In an embodiment the compound is selected from the group consisting of 5α-pregnane-3α,6α,20α-triol glucuronide, 5α-pregnane-3β,6α,20α-triol glucuronide, 5α-pregnane-3α,6β,20α-triol glucuronide, 5α-pregnane-3β,6β,20α-triol glucuronide, 5α-pregnane-3α,6α,20β-triol glucuronide, 5α-pregnane-3β,6α,20β-triol glucuronide, 5α-pregnane-3α,6β,20β-triol glucuronide, and 5α-pregnane-3β,6β,20β-triol glucuronide.

In an embodiment the compound is 5α-pregnane-3,6,20-triol-3-O-α-D-glucuronide, 5α-pregnane-3,6,20-triol-6-O-α-D-glucuronide, 5α-pregnane-3,6,20-triol-20-O-α-D-glucuronide, 5α-pregnane-3,6,20-triol-3-O-β-D-glucuronide, 5α-pregnane-3,6,20-triol-6-O-β-D-glucuronide, or 5α-pregnane-3,6,20-triol-20-O-β-D-glucuronide.

In an embodiment a combination or mixture of 5α-pregnane-3,6,20-triol isomers and/or 5α-pregnane-3,6,20-triol isomers in a glucuronidated form may be used.

The present disclosure also describes use of a compound in determination of progestogen or progestogens in a sample from a non-human animal, wherein the compound comprises 5α-pregnane-3,6,20-triol isomer or 5α-pregnane-3,6,20-triol isomer in a glucuronidated form.

In an embodiment the present disclosure describes use of a compound in determination of progestogen or progestogens in an in vitro sample from a non-human animal, wherein the compound comprises 5α-pregnane-3,6,20-triol isomer or 5α-pregnane-3,6,20-triol isomer in a glucuronidated form.

Especially, the present disclosure describes use of a compound in determination of progestogen or progestogens in an in vitro urine sample from a non-human animal, wherein the compound comprises 5α-pregnane-3,6,20-triol isomer or 5α-pregnane-3,6,20-triol isomer in a glucuronidated form.

In an embodiment the compound is 5α-pregnane-3,6,20-triol isomer.

In an embodiment the compound is 5α-pregnane-3,6,20-triol glucuronide isomer.

In an embodiment the present disclosure describes use of a compound selected from the group consisting of 5α-pregnane-3α,6α,20α-triol, 5α-pregnane-3β,6α,20α-triol, 5α-pregnane-3α,6β,20α-triol, 5α-pregnane-3β,6β,20α-triol, 5α-pregnane-3α,6α,20β-triol, 5α-pregnane-3β,6α,20β-triol, 5α-pregnane-3α,6β,20β-triol, and 5α-pregnane-3β,6β,20β-triol in determination of progestogen or progestogens in an in vitro sample from a non-human animal.

In an embodiment the present disclosure describes use of a compound selected from the group consisting of 5α-pregnane-3α,6α,20α-triol glucuronide, 5α-pregnane-3β,6α,20α-triol glucuronide, 5α-pregnane-3α,6β,20α-triol glucuronide, 5α-pregnane-3β,6β,20α-triol glucuronide, 5α-pregnane-3α,6α,20β-triol glucuronide, 5α-pregnane-3β,6α,20β-triol glucuronide, 5α-pregnane-3α,6β,20β-triol glucuronide, and 5α-pregnane-3β,6β,20β-triol glucuronide in determination of progestogen or progestogens in an in vitro sample from a non-human animal.

In an embodiment the present disclosure describes use of a compound selected from the group consisting of 5α-pregnane-3,6,20-triol-3-O-α-D-glucuronide, 5α-pregnane-3,6,20-triol-6-O-α-D-glucuronide, 5α-pregnane-3,6,20-triol-20-O-α-D-glucuronide, 5α-pregnane-3,6,20-triol-3-O-β-D-glucuronide, 5α-pregnane-3,6,20-triol-6-O-β-D-glucuronide, or 5α-pregnane-3,6,20-triol-20-O-β-D-glucuronide in determination of progestogen or progestogens in an in vitro sample from a non-human animal.

In an embodiment the sample is an excrete. In a preferred embodiment the excrete is selected from the group consisting of urine, saliva, and feces. In a more preferred embodiment, the sample is a urine sample.

In an embodiment the present disclosure describes use of the compound according to the present disclosure in determination of progestogen or progestogens in an in vitro urine sample from a non-human animal.

In an embodiment the present disclosure describes use of a compound selected from the group consisting of 5α-pregnane-3α,6α,20α-triol, 5α-pregnane-3β,6α,20α-triol, 5α-pregnane-3α,6β,20α-triol, 5α-pregnane-3β,6β,20α-triol, 5α-pregnane-3α,6α,20β-triol, 5α-pregnane-3β,6α,20β-triol, 5α-pregnane-3α,6β,20β-triol, and 5α-pregnane-3β,6β,20β-triol in determination of progestogen or progestogens in an in vitro urine sample from a non-human animal.

In an embodiment the present disclosure describes use of a compound selected from the group consisting of 5α-pregnane-3α,6α,20α-triol glucuronide, 5α-pregnane-3β,6α,20α-triol glucuronide, 5α-pregnane-3α,6β,20α-triol glucuronide, 5α-pregnane-3β,6β,20α-triol glucuronide, 5α-pregnane-3α,6α,20β-triol glucuronide, 5α-pregnane-3β,6α,20β-triol glucuronide, 5α-pregnane-3α,6β,20β-triol glucuronide, and 5α-pregnane-3β,6β,20β-triol glucuronide in determination of progestogen or progestogens in an in vitro urine sample from a non-human animal.

In an embodiment the present disclosure describes use of a compound selected from the group consisting of 5α-pregnane-3,6,20-triol-3-O-α-D-glucuronide, 5α-pregnane-3,6,20-triol-6-O-α-D-glucuronide, 5α-pregnane-3,6,20-triol-20-O-α-D-glucuronide, 5α-pregnane-3,6,20-triol-3-O-β-D-glucuronide, 5α-pregnane-3,6,20-triol-6-O-β-D-glucuronide, or 5α-pregnane-3,6,20-triol-20-O-β-D-glucuronide in determination of progestogen or progestogens in an in vitro urine sample from a non-human animal.

In an embodiment the animal is selected from the group of canines consisting of a domestic dog, wild dogs, wolves, coyotes, foxes, bush dog, short-eared dog, jackals, and raccoon dogs.

In an embodiment the sample is an in vitro sample.

The disclosure describes an in vitro method of determining progestogen or progestogens in a sample from a non-human animal, wherein the method comprises the steps of providing an in vitro sample from a non-human animal, measuring an amount of 5α-pregnane-3,6,20-triol isomer or 5α-pregnane-3,6,20-triol glucuronide isomer in said sample, determining the presence of luteinized follicular theca and/or granulosa cells based on the amount of 5α-pregnane-3,6,20-triol isomer or 5α-pregnane-3,6,20-triol glucuronide isomer in the sample, wherein the progestogen level in the sample of the non-human animal is indicative by the presence of luteinized follicular theca and/or granulosa cells.

In an embodiment the method comprises an in vitro method of determining progestogen or progestogens in a urine sample from a non-human animal, wherein the method comprises the steps of providing an in vitro urine sample from a non-human animal, measuring an amount of 5α-pregnane-3,6,20-triol isomer or 5α-pregnane-3,6,20-triol glucuronide isomer in said sample, determining the presence of luteinized follicular theca and/or granulosa cells based on the amount of 5α-pregnane-3,6,20-triol isomer or 5α-pregnane-3,6,20-triol glucuronide isomer in the sample, wherein the progestogen level in the sample of the non-human animal is indicative by the presence of luteinized follicular theca and/or granulosa cells.

In an embodiment the method comprises an in vitro method of determining progestogen or progestogens in a urine sample from a non-human animal, wherein the method comprises the steps of providing an in vitro urine sample from a non-human animal, measuring an amount of 5α-pregnane-3,6,20-triol isomer in said sample, determining the presence of luteinized follicular theca and/or granulosa cells based on the amount of 5α-pregnane-3,6,20-triol isomer in the sample, wherein the progestogen level in the sample of the non-human animal is indicative by the presence of luteinized follicular theca and/or granulosa cells.

In an embodiment the method comprises an in vitro method of determining progestogen or progestogens in a urine sample from a non-human animal, wherein the method comprises the steps of providing an in vitro urine sample from a non-human animal, measuring an amount of 5α-pregnane-3,6,20-triol glucuronide isomer in said sample, determining the presence of luteinized follicular theca and/or granulosa cells based on the amount of 5α-pregnane-3,6,20-triol glucuronide isomer in the sample, wherein the progestogen level in the sample of the non-human animal is indicative by the presence of luteinized follicular theca and/or granulosa cells.

Based on the amount of 5α-pregnane-3,6,20-triol isomer or 5α-pregnane-3,6,20-triol glucuronide isomer in a sample, conclusions can be drawn about the stage of oestrus. The presence of luteinized follicular theca and/or granulosa cells in the sample can be determined based on the amount of 5α-pregnane-3,6,20-triol or its glucuronidated form in the sample.

An animal's, such as canine's, optimal breeding time can be determined by measuring an amount of 5α-pregnane-3,6,20-triol or its glucuronidated form in an in vitro sample.

Especially, an animal's, such as canine's, optimal breeding time can be determined by measuring an amount of 5α-pregnane-3,6,20-triol or its glucuronidated form in an in vitro urine sample.

In an embodiment a combination or mixture of 5α-pregnane-3,6,20-triol isomers and/or 5α-pregnane-3,6,20-triol isomers in a glucuronidated form may be used in the method.

The present method can be used for determining the oestrus cycle phase in an animal, such as canids. An increase in the concentration of the serum progesterone hormone to a certain level indicates that ovulations occurred, as a result of which conception is possible. The progestogen level in the sample of the non-human animal is indicative by the presence of luteinized follicular theca and/or granulosa cells.

The present method of determining progestogen or progestogens is carried out by measuring an amount of 5α-pregnane-3,6,20-triol isomer or 5α-pregnane-3,6,20-triol glucuronide isomer in an in vitro urine sample of a non-human animal.

In the present method quantification of progestogen or progestogens is carried out by measuring an amount of 5α-pregnane-3,6,20-triol isomer or 5α-pregnane-3,6,20-triol glucuronide isomer in an in vitro sample of a non-human animal.

In an embodiment the animal is preferably a canine. More preferably the canine is selected from the group consisting of a domestic dog, wild dog, wolves, coyotes, foxes, a bush dog, a short-haired dog, jackals, and raccoon dog.

In an embodiment the sample is an excrete. Preferably the excrete is selected from the group consisting of urine, saliva, and feces. More preferably the sample is urine sample.

In the present method quantification of progestogen or progestogens is carried out by measuring an amount of 5α-pregnane-3,6,20-triol isomer or 5α-pregnane-3,6,20-triol glucuronide isomer in an in vitro urine sample of a non-human animal.

The quantification may be carried out using any suitable method known to a skilled person in the art. In an embodiment the quantification method is selected from the group consisting of an immunoassay, binding assay, mass spectrometry, nuclear magnetic resonance (NMR), and chromatography.

In an embodiment of the method of the present disclosure immunoassay may be used. Immunoassay is an immunochemical method based on the specific interactions between the antigens and the antibodies to which they bind. The antigen-binder can also be only part of the antibody (such as an immunoglobulin Fab domain) or a molecule other than the antibody, e.g. aptamer, affimer, protein A (and others) or receptor. Any molecule which is known to bind to a specific antibody can act as antigen. In a basic immunoassay method, one of the antigen-antibody pairs is immobilized onto the solid support as a capture agent, which is then probed by the other member of the pair. Subsequently, the captured molecules or complexes are detected using the labeled molecules, usually the antibodies.

Immunoassay-based methods have already been developed in countless numbers and more are being developed all the time. The method can be applied almost indefinitely to any suitable environment. Suitable immunoassay-based methods include for example radioimmunoassay (RIA), enzyme immunoassay (EIA, ELISA) and fluorescence immunoassay (IFA, FIA, etc.) as well as immunoelectrophoresis and lateral flow (LFIA). As a skilled person in the art is able to select a suitable method to be used.

The immunoassay formats can be classified into two types: forward-phase and reversed-phase assays, depending on which molecule of interest is involved in the assay; the capture or the probe. Furthermore, the assay formats can be divided into competitive and non-competitive categories, depending on the assay being used. Competitive assays are mainly used to assess the expression levels of small molecules, such as steroids or drugs, while non-competitive assays are generally used to measure (qualitatively or quantitatively) or analyze larger biomolecules such as proteins and antibodies.

The forward-phase assays use well-characterized molecules, with known target specificities, immobilized onto the solid support as capture agents. The capture agents are probed with a sample solution such as saliva, urine or fecal supernatant with the goal being to measure the presence, absence, or concentration of the molecule of interest. In the reversed-phase formats, antibodies are used to probe molecules immobilized on the solid phase. The analysis of the resulting binding profile can help to identify new biomarker candidates for the development of diagnostics, or it can also be used, for example, in antibody specificity screening.

Immunoassay is based on the attachment of the antigen/antibody to the solid phase. Almost any solid surface to which the desired molecule can be attached can act as the basis for immunoassay. The most common solid phase used in immunoassay is a conventional 96-well plate, but in addition e.g., microscope slides, test tubes, membranes, rapid test platforms as well as various micro-/nanoparticles and gel matrixes are used as substrates.

The most used solid support materials are plastics (e.g. polystyrene, polycarbonate, poly(methyl methacrylate), polyethylene terephthalate), glass (mainly silicon dioxide) and metals (such as gold, silver, platinum, aluminum, iron, cobalt, copper, titanium). In addition, for example, silicon, carbon, lipids, (nitro)cellulose etc. are used. The materials used for manufacturing solid support materials are almost unlimited.

Some of the solid support materials are capable of binding proteins without any pre-activation, but the assays are usually performed on functionalized surfaces. The surface modifications can be divided into two types; two-dimensional (2-D), including e.g. surface activation via plasma treatment, irradiation, or chemical treatment, and three dimensional (3-D) where a chemically activated porous layer is fabricated onto a solid support by physical or chemical methods. The 3-D surface layers are typically polymers which can form membranes, hydrogels, layers, brushes, monoliths or dendrimers. The 3-D surfaces have an increased binding capacity versus the 2-D surfaces due to their porous nature and thus their higher binding area.

The attachment of the antigen/antibody to the solid phase can be performed via physical or chemical methods or using carrier proteins. The physical binding process is based on the direct adsorption or entrapment of the proteins, e.g. they can be passively attached onto the surface through non-specific interactions. The immobilization by the direct adsorption is mainly based on hydrophobic, hydrophilic, and electrostatic binding interactions between the protein and the solid surface. In the chemical attachment, new molecular bonds are formed between the protein and the surface. The covalent binding can occur with the chemically active moieties of the proteins (usually primary amines or thiol groups) and with the surface of the solid support patterned with chemically active groups like a primary amine, carboxylic acid, aldehyde, or epoxide.

Proteins are widely used as carriers to increase binding affinity and to perform site-specific immobilization of molecules with only one antigenic site, or they are not capable to immobilization onto solid support. The use of the protein mediated immobilization requires a bond formation between the carrier protein and the capture agent. This can be performed either via direct conjugation of the capture agent onto the carrier protein by covalent linking or via affinity binding by non-covalent, specific recognition.

Examples of the carrier proteins (for covalent linking) in immunoassays are serum albumin obtained from either cows (BSA) or humans (HSA) and ovalbumin protein from avian egg-white, but also other proteins have been exploited. The protein carriers are widely used for the immobilization of small molecular weight chemical compounds, such as steroids, toxins, and drugs, which are not able to bind onto a solid support without losing their antigenic properties. In addition, the requirement to use a carrier protein is often related to the need to elicit an immune response in the development of antibodies and vaccines, for example, and thus it is necessary to analyze the haptens. Widely used immunogenic carrier proteins are BSA, ovalbumin, keyhole limpet hemocyanin (KLH) and blue carrier protein (mollusk-derived hemocyanin).

In an embodiment of the method of the present disclosure a binding assay may be used.

Affinity binding refers to a non-covalent specific recognition-based bond between two different molecules. The most extensively used affinity binding methods are the exploitation of the specific binding properties of antibodies and the biotin-avidin (or streptavidin/neutravidin) interaction. Antibody mediated affinity binding is mainly used for the immobilization of antibodies in an oriented form. Oriented immobilization exposes the antigen-binding sites, thus significantly improving the target binding efficiency and detection sensitivity. Antibodies can be utilized as capture agents also by non-oriented immobilization, especially monoclonal antibodies which possess a certain type of binding sites. If antisera or polyclonal antibodies, which bind to multiple epitopes, are used as capture agents, the affinity binding via a secondary antibody system can be applied to increase the binding affinity of specific antibodies towards the antigens of interest. The antibodies can also bind other capture agents, such as glycoproteins.

Many different methods are used to detect the formed antigen-antibody complex in an immunoassay, and more are created all the time. Conventionally the desired antigen or antibody conjugated (directly or using affinity binding tags) with some label is used for the detection. In immunological assays, the most widely used labels are enzymes such as horseradish peroxidase (HRP) and alkaline phosphatase, which function either by producing a detectable color change (colorimetric) or precipitation, or by producing light or chemiluminescence when exposed to some reagents. The widely used labels are also radioactive isotopes and fluorescent chemical compounds, but also other methods such DNA-reporters and various detectable nano-/microparticles (e.g. precipitation or turbidimetric) have been described. In addition, immunoassay detection can be performed also without the label using surface plasmon resonance technique or measuring the change in resistance on an electrode as antigens bind to it.

Progestogen metabolites can be measured by various immunological assays. In the present method, either polyclonal or monoclonal antibodies raised against 5α-pregnane-3,6,20-triol isomer or 5α-pregnane-3,6,20-triol glucuronide isomer can be used as primary antibodies.

In an embodiment of the present disclosure an immunoassay is selected from the group consisting of an enzyme immunoassay (ELISA, EIA), radioimmunoassay (RIA), fluorescence immunoassay, immunoelectrophoresis, lateral flow immunoassay, electrochemiluminescence assay, and nanoparticle-based assay. A skilled person is able to select a suitable immunoassay method for use in detection.

In an embodiment of the present disclosure a rapid test is used for determination. In a typical semi-quantitative or quantitative lateral flow immunochromatographic assay urinary progestogen metabolite concentration is measured. In an example of such an assay, urinary progestogen metabolites are competing with progestogen metabolites conjugated to gold nanoparticles. A skilled person is able to select a suitable format for a rapid test.

In an embodiment of the present disclosure urinary 5α-PtG concentration differences between oestrus cycle phases in a dog are tested by lateral flow chromatography. This rapid test provides a quick and cost-effective method to discover the urinary progestogen levels in a dog without the need for specialized equipment or laboratory facilities.

In an embodiment of the present disclosure, the rapid test is a lateral flow immunochromatographic test strip, which includes a sample pad, conjugate pad, nitrocellulose membrane and absorbent pad. A skilled person is able to select suitable materials to be included in the rapid test. An example of the test strip is presented in FIG. 29. The buffering reagents are absorbed into the sample pad to smooth out the variation between samples due to, for example, variation in pH and salt concentration. The antibody, such as 5α-PtG antibody, conjugated to the gold particle is dried on the conjugate pad. The conjugate, such as 5α-PtG conjugate, is immobilized to the nitrocellulose membrane as a test line and anti-mouse IgG antibody as a control line. The excess sample is eventually absorbed into the absorbent pad, which also contributes to the capillary forces on the test strip.

In an embodiment of the present disclosure, in the rapid test, the sample is placed on a sample pad and the sample flows through the test strip by capillary forces. On the conjugate pad, the analyte molecule, such as 5α-PtG molecule, contained in the sample binds to the antibody, such as 5α-PtG molecule, conjugated to gold nanoparticle, and the formed complex moves to the membrane. If only a small amount of the analyte, such as 5α-PtG, is present in the sample, the free antibody-gold nanoparticles will flow through the membrane and bind to the analyte, such as 5α-PtG-BSA, in the test line, producing a visible signal. When the analyte molecules, such as 5α-PtG, in the sample bind to the flowing antibody-gold nanoparticles, the antibodies do not interact with the analyte conjugate, such as 5α-PtG-BSA conjugate, and no visible test line is formed. Remained antibody-gold particles will bind to the anti-mouse IgG in the control line, producing a visible control line confirming that the fluid has successfully passed through the sample pad and test line to the control line. As the intensity of the test line is dependent on the amount of antigen present in the sample, the result, e.g., the estimation of the oestrus cycle phase can be accomplished by simple visual evaluation.

In another embodiment of the method measuring the amount of 5α-pregnane-3,6,20-triol isomer or 5α-pregnane-3,6,20-triol glucuronide isomer in an in vitro sample from a non-human animal comprises contacting the sample with a capture moiety and a detection moiety, wherein the capture moiety is 5α-pregnane-3,6,20-triol or 5α-pregnane-3,6,20-triol glucuronide isomer attached to a solid support as conjugated with a carrier protein, and the capture moiety is capable of forming a complex with an antibody against 5α-pregnane-3,6,20-triol or 5α-pregnane-3,6,20-triol glucuronide isomer; wherein the detection moiety is capable of binding to the antibody of the antibody-capture moiety complex; wherein the formation of the complex is indicative of the presence of luteinized follicular theca and/or granulosa cells in a non-human animal; detecting a signal from the detection moiety, wherein the amount of signal detected is proportional to the oestrus cycle phase. In a preferred embodiment, the sample is urine. In a preferred embodiment, the non-human animal is selected from the group of canines consisting of a domestic dog, wild dogs, wolves, coyotes, foxes, a bush dog, a short-haired dog, jackals, and raccoon dogs.

In an embodiment the detection moiety comprises a binding partner and a label, and wherein the label is selected from the group consisting of electrochemiluminescence labels or compounds, chemiluminescent compounds, enzyme labels, fluorophores, chromogenic compounds, radiolabels, catalysts, a latex particles, a magnetic particles, a radioactive elements, fluorescent dyes, phosphorescent dyes, dye crystallites, gold colloidal particles, silver colloidal particles, selenium colloidal particles, metal chelates, coenzymes, electro active groups, oligonucleotides, and stable radicals.

In an embodiment of the present disclosure the solid support comprises a bead, a superparamagnetic bead, a paramagnetic bead, a plate, a glass surface, a plastic surface, a metal surface, a polystyrene surface, a nitrocellulose surface, sepharose, agars, a microparticle surface, a nanoparticle surface, a flow path in a lateral flow assay device, or a well in a microtiter plate.

In an embodiment of the method of the present disclosure chromatography may be used. In chromatography, an analyte of interest is separated from the mixture of compounds, e.g. body fluids. Chromatography is based on interactions between stationary phase and mobile phase, in which the mixture is dissolved in (Beesley and Buglio, 2000).

Chromatography may be used for preparative (separation, purification) or analytical purposes (quantitative and qualitative analysis). In general, the separation is based on the structure and physiochemical properties of the analyte, including size (size-exclusion chromatography, SEC), polarity, respective charge (ion chromatography) and adsorption (expanded bed-chromatographic adsorption, EBA). The chromatographic techniques are usually named by the bed shape (column and planar chromatography), properties of mobile phase (gas and liquid chromatography) or separation mechanism (e.g. affinity, ion exchange, size-exclusion, EBA). Chromatography is commonly used with measuring devices such as mass spectrometer or UV detectors.

For analytical measurements, samples are prepared for analysis. The efficient sample preparation is repeatable and has high recovery of the analyte. Preparation of the samples might be necessary before the chromatography to minimize the matrix effect of the sample, but various chromatographic methods can also be used in sample preparation. Body fluids as blood, saliva, tissue fluids and excretions must be homogenized and purified from solid particles. The extraction of the steroids removes the undesired compounds from the sample, which might interfere with the analysis. Ultra-centrifugation, precipitation and dialysis are cost-effective methods to remove or isolate high-molecular weighted compounds as proteins from the sample. In exclusion chromatography, large compounds are removed from the sample matrix by eluting the sample through the stationary phase, which pore size is appropriate to analyte of interest. Solid-phase extraction (SPE) is a rapid method for sample preparation and purification. In the SPE, complex matrix as urine or blood, can be easily purified by normal phase, reverse-phase or by ion exchange extraction, depending on the packing type of the stationary phase. SPE provides efficient techniques to prepare samples prior the chromatographic analysis.

The sample preparation is dependent on the chromatographic method. For example, in liquid chromatography the analyte of interest is usually either diluted with proper solvent as acetonitrile or steroids are extracted by liquid-liquid extraction before chromatography. For gas chromatography (GC), the volatility of naturally non-volatile steroids is increased be derivatization. The advances of gas chromatography are high sensitivity of GC detectors and high efficiency, which is why GC provide accurate and precise results. In addition, the spectrum libraries of derivatized steroids are comprehensive. In liquid chromatography (LC), the steroids can be detected in their nature form and derivatization is not needed. Like GC, LC is considered as an accurate and precise method for analysis. A skilled person can select a suitable chromatography method.

In an embodiment the chromatography is selected from the group consisting of gas chromatography, gas chromatography-mass spectrometry, liquid chromatography, and liquid chromatography-mass spectrometry.

In an embodiment the progesterone originated metabolites can be identified and measured by liquid chromatography—high resolution mass spectrometry (LC-HRMS) method. The level of metabolites can be used as a biomarker of circulating progestogens.

In an embodiment of the method of the present disclosure mass spectrometry may be used. Mass spectrometry (MS) is a tool for many areas of research. The MS is a highly sensitive and versatile analytical technique used to identify and quantify molecules in various sample types, including biological samples such as urine, saliva, and blood. It works by ionizing a sample, separating the ions based on their mass-to-charge ratio (m/z), and detecting them to determine the molecular weight and elemental composition.

In ionization, analytes are transformed into ions. Negatively or positively charged ions are accelerated in an electric field, extracted, and directed to a mass analyzer and detector, providing information about the molecular composition of the sample. The ionization can be accomplished by various methods, including electron ionization (EI), electrospray ionization (ESI), chemical ionization (CI), matrix-assisted laser desorption ionization (MALDI), atmospheric-pressure chemical ionization (APCI), and many more. The EI involves removing electrons from the sample molecules to create positive ions, while the CI involves reacting the sample with a reagent gas to create ions. The MALDI uses a laser to ionize the sample. In ionization, the analyte will be fragmented, and the fragmentation degree is dependent on the energy in the ionization. In hard ionization (as in EI), the degree of fragmentation is high, whereas soft ionization (as in ESI, CI, MALDI, APCI) results in only minor fragmentation. Soft ionization provides m/z close to the neutral mass of the analyte.

Mass analyzers are used to separate the ions based on their m/z. Mass analyzers use either static or dynamic fields and magnetic or electric fields for separation. For example, time-of-flight (TOF) and quadrupole mass analyzers use electric fields to accelerate the ions. In tandem mass spectrometry (MS/MS), two or more analyzers are used, providing more accurate information about the ions and fragmentation.

A time-of-flight (TOF) analyzer is a mass spectrometer that separates ions based on the time it takes to travel a set distance. It works by accelerating the ions and measuring the time it takes for them to reach a detector at the end of a flight tube. The time it takes for the ions to reach the detector is proportional to their mass-to-charge ratio, so the TOF analyzer can separate and identify the ions based on their mass.

A quadrupole mass analyzer is a type of mass spectrometer that uses four parallel rods to focus and analyze ions based on their mass-to-charge ratio. The rods create an alternating potential that acts as a mass filter. Ions with the desired mass-to-charge ratio can pass through the quadrupole and be detected, while ions with other mass-to-charge ratios are deflected and do not reach the detector.

Orbitrap is a mass spectrometer that uses a combination of an electric and magnetic field to analyze ions based on their mass-to-charge ratio. The ions are trapped in a toroidal-shaped electric field and their motion is analyzed as they orbit around the center of the field. The frequency of the ion's motion is proportional to its mass-to-charge ratio, and this information is used to determine the mass of the ion.

A mass detector is a device used in mass spectrometry to measure the mass of ions. It works by measuring the ion's response to an electric or magnetic field, and the information collected is used to determine the ion's mass-to-charge ratio. The most used mass detectors in mass spectrometry include the quadrupole mass filter, the time-of-flight (TOF) analyzer, and the Orbitrap. Each type of mass detector has unique advantages and disadvantages, and the choice of the detector depends on the specific application and requirements of the analysis. Mass detectors are an essential component of mass spectrometers and play a critical role in analyzing and identifying compounds in a sample.

Mass spectrometry is widely used in fields such as chemistry, biology, and health sciences for applications such as protein and metabolite analysis, trace contaminant analysis, and drug development. It offers many advantages such as high sensitivity, accuracy, and the ability to handle complex samples. Mass spectrometry provides a tool for the quantitation and identification of unknown compounds.

In an embodiment of the method of the present disclosure immunoaffinity chromatography (IAC) coupled with liquid/gas chromatography-mass spectrometry may be used. Coupled IAC-LCMS and IAC-GCMS have benefits of structure-specific antibodies and high accuracy analysis. These methods are effective to extract and concentrate the compound of interest from complex sample matrix as urine samples and analyze it by high accuracy instruments. This method can be used e.g. on low-concentrate samples when results cannot be achieved without efficient concentrating methods or when subsequent analyzing/preparative methods demand efficient purification process.

In an embodiment of the method of the present disclosure nuclear magnetic resonance (NMR) spectroscopy may be used. NMR is an analytical technique to determine structures of chemical and biological compounds. It relies on the interaction of the nuclei of certain atomic isotopes with a static magnetic field, and detects the energy absorbed by changes in the nuclear spin state. The most used nuclei are ¹H and ¹³C, but isotopes of other elements, such as ¹⁹F and ³¹P can be studied as well. There are one-dimensional and two-dimensional techniques, of which the latter are sometimes needed when determining the structure of more complicated molecules. NMR experiments are typically performed in solution.

Atomic nuclei have intrinsic spin properties and different atoms in a molecule differ in resonance frequency in the same magnetic field, a phenomenon known as the chemical shift (δ). It means that in an NMR spectrum, different atoms give different peaks according to specific chemical environments and bonding between atoms. In addition, the resonance frequencies are perturbed by neighboring NMR active nuclei, dependent on the bonding electrons connecting the nuclei, which means that connections between atoms on a molecule can be identified. The observable NMR signal is recorded by the spectrometer as free induction decay (FID) which is then Fourier transformed, resulting in a spectrum consisting of a set of peaks in which each peak corresponds to a distinct chemical environment. The number of nuclei in a specific chemical environment is directly related to the area underneath the peak. The chemical shifts are expressed in parts per million (ppm).

NMR experiments are performed in deuterated solvents to avoid solvent interference, to stabilize the field strength and to accurately define 0 ppm. Comparisons to reference spectra should be made with same solvents since the peaks in the spectra can be solvent dependent. The most common solvents are deuterated chloroform, acetone, dimethyl sulfoxide, acetonitrile, methanol, and water.

In an embodiment of the present disclosure an in vitro method comprises the steps of providing an in vitro urine sample from a canine, measuring an amount of 5α-pregnane-3,6,20-triol isomer or 5α-pregnane-3,6,20-triol glucuronide isomer in said sample, determining the presence of luteinized follicular theca and/or granulosa cells based on the amount of 5α-pregnane-3,6,20-triol isomer or 5α-pregnane-3,6,20-triol glucuronide isomer in the sample, wherein the progestogen level in the sample is indicative by the presence of luteinized follicular theca and/or granulosa cells.

The oestrus cycle phase in a canine is determined by urinary measurements of progestogen metabolites, 5α-pregnane-3,6,20-triol isomer or 5α-pregnane-3,6,20-triol glucuronide isomer. Thus, the optimal breeding time (mating or inseminating) in a bitch is detected.

The present disclosure describes a kit of determining progestogen or progestogens in a urine sample from a non-human animal, wherein the kit comprises immunoassay means for measuring an amount of 5α-pregnane-3,6,20-triol isomer or 5α-pregnane-3,6,20-triol glucuronide isomer in an in vitro urine sample from a non-human animal, and instructions for determining progestogen or progestogens according to the method which comprises:
a) providing an in vitro urine sample from a non-human animal,
b) measuring an amount of 5α-pregnane-3,6,20-triol isomer or 5α-pregnane-3,6,20-triol glucuronide isomer in said sample,
c) determining the presence of luteinized follicular theca and/or granulosa cells based on the amount of 5α-pregnane-3,6,20-triol isomer or 5α-pregnane-3,6,20-triol glucuronide isomer in the sample,
   wherein the progestogen level in the sample is indicative by the presence of luteinized follicular theca and/or granulosa cells.

It is apparent to a person skilled in the art that as technology advances, the basic idea of the invention can be implemented in various ways. The invention and its embodiments are therefore not restricted to the above examples, but they may vary within the scope of the claims.

### EXAMPLES

### Example 1

### Discovery of New Biomarker for Oestrus Monitoring

Progesterone originated metabolites were discovered by liquid chromatography-high resolution mass spectrometry (LC-HRMS) method. Metabolites with neutral mass of 512 can be used as a biomarker of circulating progestogens.

### Collection and Preparation of Serum Samples

The serum samples (177 samples from 22 bitches) were collected from proestrus to oestrus and provided by Vetcare Ltd. The serum samples were collected during a study in South Africa (ClinVet International (Pty) Ltd) in 2015. The study plan CW 14/122 was approved by the ClinVet Committee for Animal Ethics and Welfare (CCAEW). Serum samples were stored frozen (−20° C. or under) until analysis. In total of 95 serum samples of randomly chosen eight bitches were prepared and analyzed as mentioned in Häkkinen et al. (2018), excluding the sample dilution. Prior to analysis, serum samples were slowly thawed to room temperature and diluted 1:5 in 0.9% NaCl solution (9 mg/ml NaCl solution for injection, Braun Medical Ltd, Finland).

### Collection and Preparation of Urine Samples

Urine sample of the bitch was collected from spontaneous urination to clean container by volunteered dog owners. The sample was transferred to sample tube and frozen and kept frozen until analysis. The urine samples were slowly thawed to room temperature, mixed well and centrifuged (2-5 minutes 13 400 rpm) right before use. The urine samples parallel to serum samples were collected by ClinVet International (Pty) Ltd. The urine samples were stored frozen until analysis. To compare the differences between a dog and a human, one urine sample was collected from a volunteered woman in mid-pregnancy. Informed written consent was obtained from a participant.

### Urinary Creatinine Measurements by Jaffe's Reaction

Urinary creatinine concentrations were measured by colorimetric method based on Jaffe's reaction. Creatinine (Merck, Darmstadt, Germany) was dissolved in ultrapure water in 0.5 mg/ml concentration at the day of the assay. Standard dilution series was prepared by diluting the stock solution in total of seven times in 1:2 ratio (0.5-0.008 mg/ml). Water was used as a blank control. Centrifuged (13 400 rpm, 1.5 minutes) urine samples were diluted 1:40 in ultrapure water. Standards and diluted urine samples were added as in two replicates 10 μl per well in 96 well plates (655101; Greiner Bio-one GmbH, Frickenhausen, Germany), followed by 100 μl of picric acid solution (ten parts of 4.5 mM picric acid and one part of 1.4 M sodium hydroxide (Reagena Ltd, Siilinjärvi, Finland)). Absorbances at 490 nm were measured after 30 minutes.

Urinary creatinine concentrations were calculated by linear regression analysis (y=kx+b) and result was multiplied by diluting factor (40). Creatinine concentrations were used to normalize the steroid hormone concentrations (ng/Crmg, pmol/μmol Cr or EIC area/Cr).

$$Concentration ( \frac{mg}{ml} ) = 40 * \frac{y ( OD 490 ) - b}{k}$$

### Sample Preparation by Solid-Phase Extraction (SPE)

Glucuronidated steroid hormones were extracted from urine samples by solid-phase extraction. Silica-based C18 solid reversed-phase extraction column (Waters Sep-Pak C18 3 cc Vac Cartridge, 500 mg Sorbent per Cartridge, 55-105 μm (WAT020805)) was conditioned by draining 4 ml of methanol, followed by 6 ml of 10% methanol in ultrapure water. The sample (1 to 3 milliliters) was diluted in water or 10% methanol in 1:3 ratio and loaded to column. The column was rinsed with 3 ml of 10% methanol in ultrapure water. The flow-through liquid was disposed. The steroid glucuronides were eluted with 6 ml 50% methanol in ultrapure water to glass tube. The sample tube was inserted to heat block (+50° C.) and solvent was evaporated to dryness in nitrogen evaporator. The sample was dissolved in methanol or acetonitrile. Sample was them diluted in 30% methanol in ultrapure water (total volume 100 μl) to desired dilution in and placed in glass vial insert.

### Simple Sample Preparation

One part of supernatant was reconstituted two parts of ultrapure water, followed by addition of three parts of appropriate solvent, as HPLC ultra-gradient grade methanol or acetonitrile. The mixture was filtered by filtration plate (Captiva 96-well filter plate, 0.2 μm pore size, polypropylene filter, Agilent Technologies) to 96 deep well plates or by syringe filter (20 μm pore size) to HPLC vial. Well plates are covered with silicone well cap (Thermo Nunc 276002), vials were sealed with red PTFE/silicone screw caps and stored in +4° C. till the analysis.

Urine samples of several bitches in estrus or diestrus phase were thawed to room temperature, vigorously vortexed and centrifuged 2-5 min 13 400 rpm. Supernatants were diluted 1:3 in ultrapure water and mixed with acetonitrile 1:1 (diluted sample:ACN). The urine sample solution was filtered through either 96 well plate by centrifugation (2300 rpm, 5 min) to 1 ml deep well plates and sealed by silicone well cap or syringe filter (0.2 μm) to glass vials. 30% ACN was used as blank control. Freshly prepared samples were stored in the refrigerator until the analysis.

### Glucuronidation of Reference Steroids

Steroids were dissolved in ethanol or methanol to 1-5 mg/ml concentration and 5 μg of steroid (1.9-2 nmol) was used in reactions. Reactions were performed in following conditions: 0.1 M Tris-HCl, 4 mM MgCl, 1 mM UDPGA (uridine-5′diphosphate-glucuronic acid ammonium salt, U5625, Sigma-Aldrich, Missouri, USA) and 100 μg of protein (untreated female beagle dog liver microsomes, “CLM”, 20 mg/ml, D1500, Xenotech, Kansas, USA). The reaction solutions were kept on ice until use. Canine liver microsomes (CLM) were slowly thawed on ice. The reagents were added to reaction tubes and reaction was initiated by adding the canine microsomes. Reaction tubes were incubated at plate shaker at +37° C. for one hour and reaction was stopped by adding 300 μl of acetonitrile (ACN, high purity HPLC grade, VWR Chemicals). Reaction solutions were centrifuged 13 400 rpm for 10 minutes (Eppendorf Centrifuge 5415 D, Hamburg, Germany) and supernatant was collected and stored −20° C. until analysis.

### Non-Targeted and Semi-Quantitative Analysis of Pregnanediols and Pregnanetriols by LC-HRMS

In semi-quantitative analysis of progesterone metabolites, various steroid glucuronides were used as reference steroids. Steroids were diluted at 1-5 μM concentration in 30% acetonitrile. Thawed urine samples were prepared as described previously. 30% acetonitrile was used as blank control. Freshly prepared samples were stored in the refrigerator until the analysis.

Semi-quantitative analysis of progestogen glucuronides was carried by UHPLC-QTOF-MS system (Agilent Technologies 1290 LC, 6540 MS, Agilent Technologies, Santa Clara, CA, USA) as described by Pekkinen et al. (2013). Prepared samples were analyzed by reversed-phase chromatography on Zorbax Eclipse XDB-C18 column (100 mm×2.1 mm, 1.8 μm; Agilent Technologies, column temperature +50° C., flow rate 0.4 ml/min) with negative and positive mode ESI. The analysis was performed by gradient elution with ultrapure water (eluent A) and methanol (eluent B) containing 0.1% (V/V) of formic acid: 0-10 min: 2→100% B; 10-14.5 min: 100% B; 14.51-16.5 min: 2% B. The injection volume of samples was 2 μl and samples were maintained at +4° C. The MS ion source conditions and MS2 analyses are described in Table 3. Also, a similar method with Thermo Scientific Q Exactive Quadrupole-Orbitrap high resolution MS/MS system with Thermo Scientific Vanquish Binary Flex Binary 1000 bar UHPLC system was used.

TABLE 3
MS ion source conditions and MS2 analysis information.

| | |
|---|---|
| Drying gas temperature | 325° C. |
| Drying gas flow | 10 l/min |
| Sheath gas temperature | 350° C. |
| Sheat gas flow | 11 l/min |
| Debulizer pressure | 45 psi |
| Capillary voltage | 3500 V |
| Nozzle voltage | 1000 V |
| Mass range | 20-1600 amu |
| Scan speed | 600 ms |
| Continuous mass axis calibration | yes |
| Number of reference ions in axis | 2 |
| calibration (infusion solution) | |
| Quadrupole isolation width | 1,3 amu |
| Collision energy | 10, 20 and 40 V |
| Number of most abundant ios | 4 |
| selected for fragmentation | |

### Results

Data analysis was performed with MassHunter Acquisition B.06.00 or newer version (Agilent Technologies, Santa Clara, CA, USA). Pregnanediol and pregnanetriol glucuronides (PdG and PtG) were analyzed from extracted ion chromatograms (EIC). The PdGs are found at 495 [M-H]−, 514 [M+NH4]+ and 519 [M+Na]+. The PtGs are found at m/z 511 [M-H]−, m/z 530 [M+NH4]+ and m/z 535 [M+Na]+.

Two synthetic pregnanediols (5β-pregnane-3α,20α-diol and 5β-pregnane-3α,20β-diol) were conjugated with glucuronic acid by CLM, and products were compared with the commercially available PdG (5β-pregnane-3α,20α-diol-3-glucuronide, later PdGα) and urine samples of a dogs in diestrus phase. Mass spectrometric analysis showed that progestogens are excreted to urine as several glucuronidated pregnanediols, -triols and -tetrols. Many of these metabolites remain unidentified, but interestingly results suggested that the β-reduction of carbonyl in C20 is highly preferred against α-reduction.

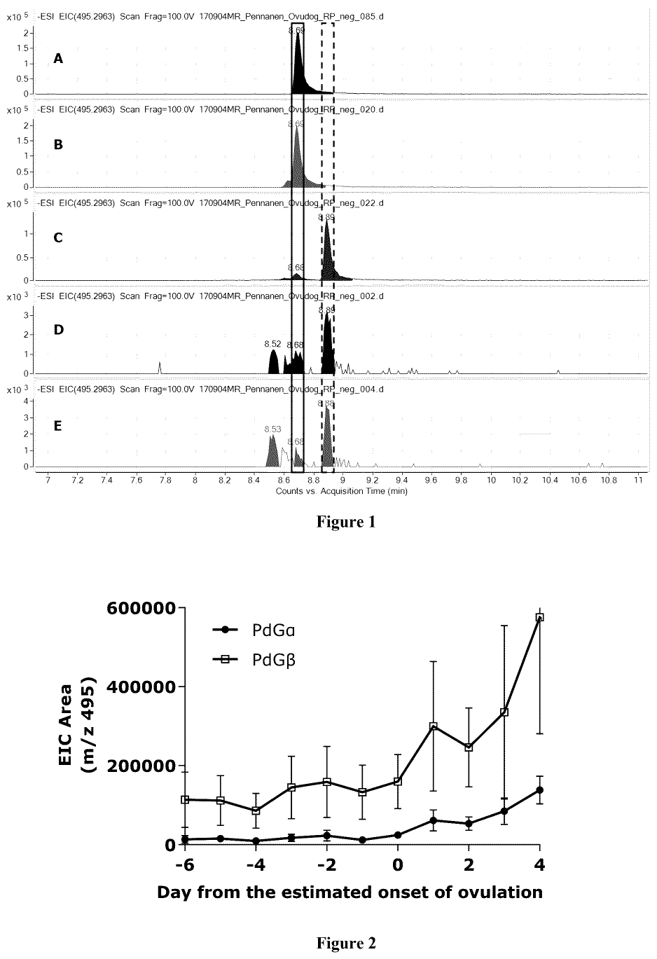

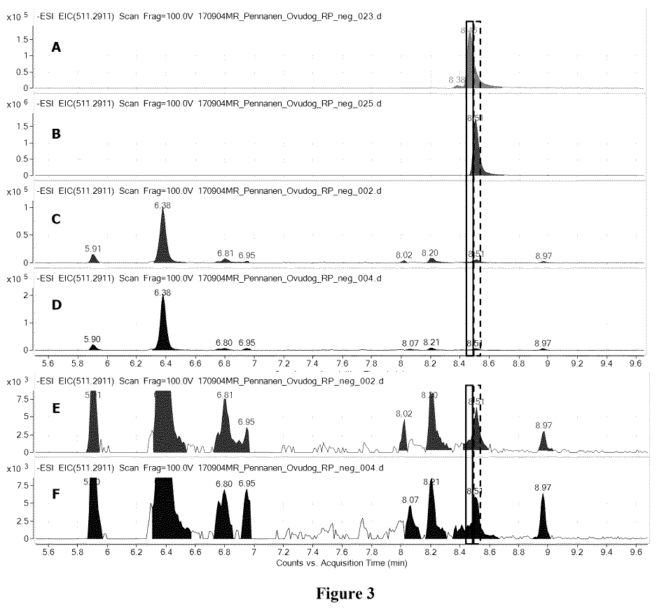

According to these results, the PdG (m/z 495 [M-H]−) was found only in traces. The semi-quantitative analysis reveals that only small amount of the PdGα and 5β-pregnane-3α,20β-diol glucuronide (later PdGβ) can be detected in urine samples (FIG. 1). The PdG is excreted mostly in the 20β-form; the average ratio between PdGβ and PdGα was 5.0 (1.0-14.7; FIG. 2).

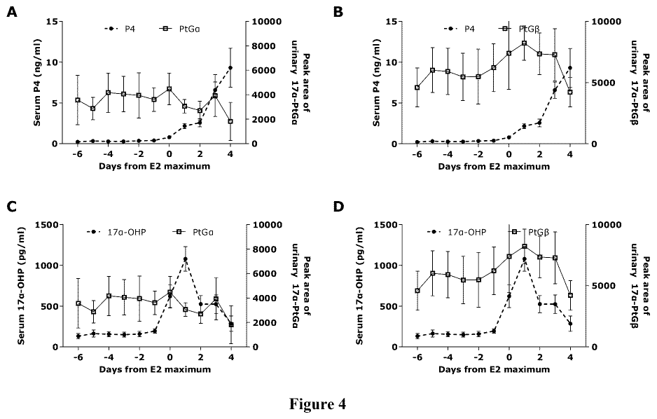

Glucuronidated synthetic pregnanetriols were compared with pregnanetriol glucuronides of urine samples collected from two bitches in diestrus phase (m/z 511 [M-H]−, m/z 530 [M+NH4]+, m/z 535 [M+Na]+). According to the literature, the common metabolite of progesterone and/or 17α-hydroxyprogesterone is 5β-pregnane-3α,17α,20α-triol-3-glucuronide (later 17α-PtGα). In dogs, the carbonyl in C20 is preferably reduced to 20β-form instead of 20α-form (FIG. 3). The urinary 17α-PtGs were compared with serum progesterone and 17α-OHP concentrations. Neither of the metabolites follow the profile of the serum progesterone (FIG. 4, a-b) or the serum 17α-hydroxyprogesterone (FIG. 4, c-d) and thus could not be used as biomarkers for oestrus phases. However, there was a significant amount of previously unidentified pregnanetriol glucuronide (later PtGX) at retention time 6.4-6.7 minutes (FIG. 3). The retention times did slightly vary between runs and equipment.

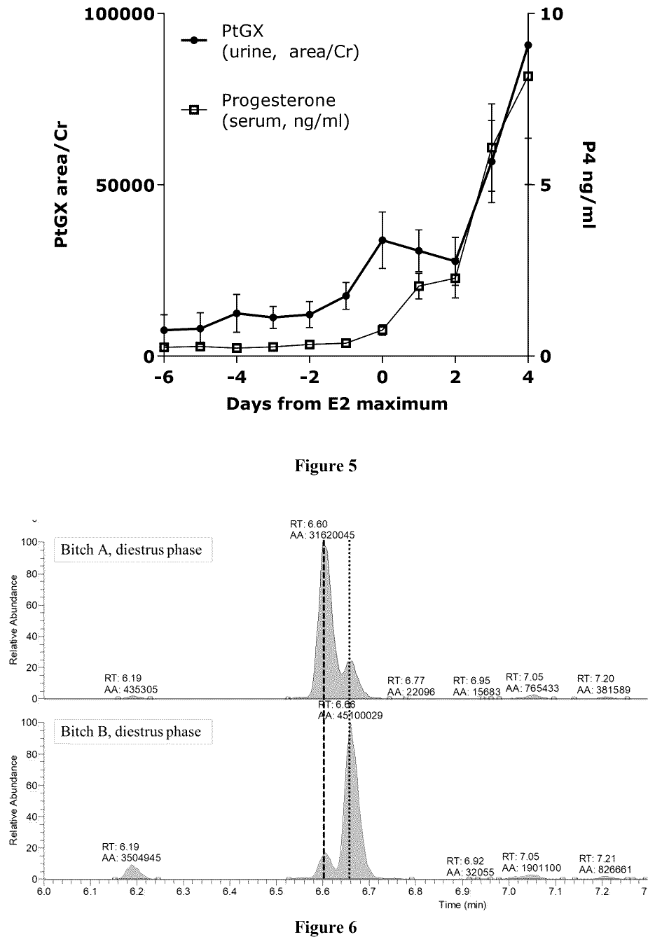

The urinary PtGX profile was similar to serum progesterone profile, which suggests that this metabolite could be used as a biomarker for oestrus phases in a similar way to serum progesterone (FIG. 5). Mass spectrometric analysis by similar method with Thermo Scientific Q Exactive Quadrupole-Orbitrap high resolution MS/MS revealed, that the PtGX was actually two close-eluting isomers at retention time 6.4-6.7 minutes (FIG. 6). In the bitch A, the most abundant PtG (later PtG1) was found 0.05 minutes earlier than the most abundant isomer in bitch B (later PtG2). However, both metabolites are found in both bitches and both metabolites can be used in oestrus monitoring as PtGX with the QTOF instrument.

### Example 2

### Comparing the Progestogen Originated Metabolites in a Dog and Human

The progestogen originated metabolites were investigated and compared by liquid chromatography-high resolution mass spectrometry (LC-HRMS) method.

### Collection and Preparation of Urine Samples

The collection of urine samples of dogs is described in Example 1. To compare the differences between a dog and a human, one urine sample was collected from a volunteered woman in mid-pregnancy. Informed written consent was obtained from a participant. The urine samples were prepared as described in Example 1 (simple sample preparation).

### Non-Targeted and Semi-Quantitative Analysis of Progestogen Originated Metabolites by LC-HRMS

Semi-quantitative analysis of progestogen glucuronides was carried by UHPLC-QTOF-MS system (Agilent Technologies 1290 LC, 6540 MS, Agilent Technologies, Santa Clara, CA, USA; negative ionization) as described in Example 1. The extracted ion chromatograms (m/z 511, 509, 507, 505 and 495) of samples were compared with each other.

### Results

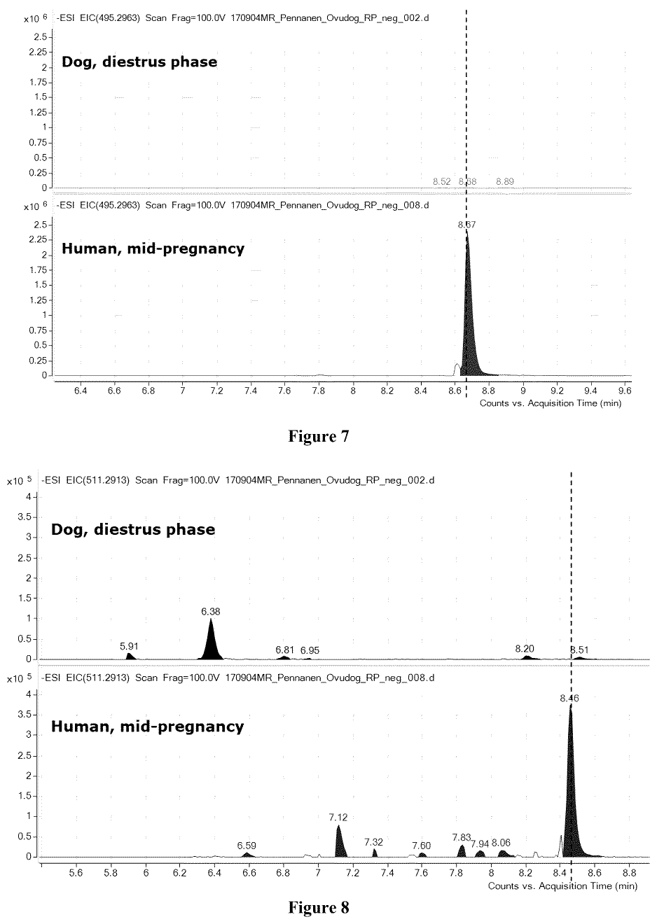

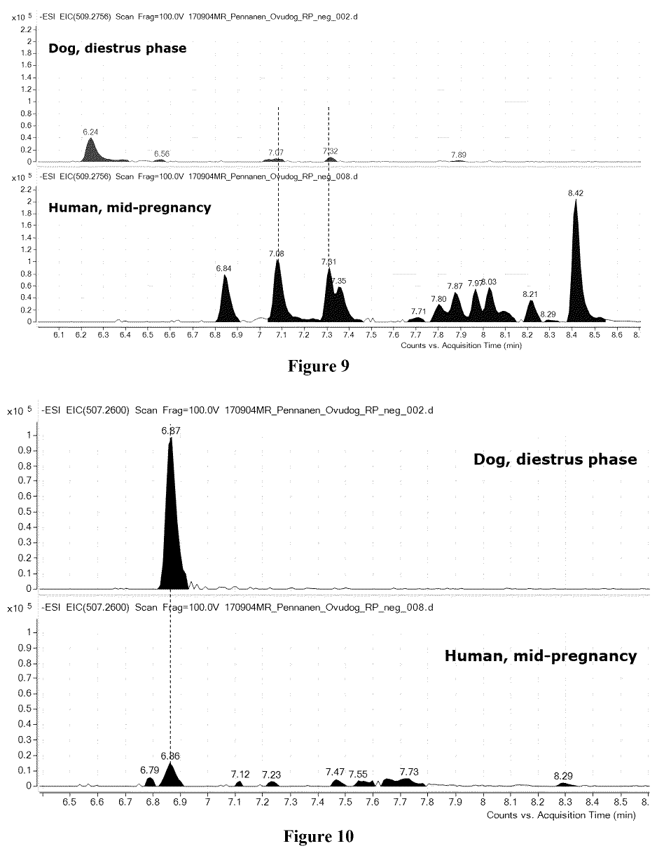

Data analysis was performed with MassHunter Acquisition B.06.00 or newer version (Agilent Technologies, Santa Clara, CA, USA). Ions equivalent with the PdG (m/z 495) were found during retention time 6.9-9.0 minutes (FIG. 7). As expected, the most abundant progesterone metabolite in human urine was found to be the PdG, whereas in dogs only traces of the PdG were found. Ions corresponding to pregnanetriol glucuronides (m/z 511) were found at RT 5.9-8.5 minutes. Human urine sample contain large amount of the 17α-PtG (FIG. 8). Both analyzed urine samples contain several unidentified pregnanetriol glucuronides (m/z 511). According to Eriksson and Gustafsson (1970), urine from pregnant woman contains at least 5β-pregnane-3α,17α,20α-triol, pregnane-3,16α,20-triol and 5α-pregnane-3,20α,21-triols. Metabolites with m/z 509 (as pregnanediol-ones) and 507 (as pregnane-ol-diones) are shown in FIGS. 9 and 10. It is clearly seen that the progesterone metabolism between a dog and a human differ significantly with each other.

### Example 3

### Identification of Progestogen Metabolite 5α-Pregnane-3,6,20-Triol

The progesterone metabolites were identified by investigating the production of hydroxyprogesterone metabolism in ovaries and liver in vitro, as well as by synthetizing new reference steroids for biomarker comparison.

### Ovarian and Hepatic Metabolism of Progesterone In Vitro

Ovaries from one bitch in anestrus phase and one bitch in diestrus phase were removed surgically during routine sterilization in veterinary clinics. Written permission for the scientific usage of the ovaries was asked from dog owners and no more harm was caused to the dogs. After removal, ovaries detached from the bursa and were frozen in −20° C. (transferred to −80° C. in 24 hours) or in liquid nitrogen (transferred to −80° C. in a few weeks) depending on the resources of the veterinary clinic. Frozen dog ovaries were weighed and cut into smaller pieces on ice, and pieces were inserted to centrifugation tube with ice-bold homogenization buffer (0.1 M Tris-HCl, 1 mM K₂EDTA, pH 7.4) and kept in ice during homogenization. The pieces were roughly homogenized with short bursts in disperser (Ultra Turrax®, Janke & Kunkel IKA-Werke TP 18/10, 20 000 rpm). The volume of homogenization buffer was four times to mass of the ovary (4 mls per 1 g of tissue). Rough homogenate was transferred in 5 ml batches to homogenization tube, kept in ice and homogenized thoroughly (Heidolph overhead stirrer RZR 2, Germany). Homogenate was divided to Beckman Coulter's tubes and centrifugated at 10 000 g for 15 minutes. Supernatant, the S9 fraction, was transferred to Eppendorf tubes and kept frozen (≤−70° C.) until use. After use, the disposal of the residual tissues is done by the waste treatment protocols of University of Eastern Finland.

In vitro progesterone hormone metabolic reactions were performed in 100 μl volume. Progesterone was dissolved in ethanol to 5 mg/ml concentration and 5 μg of steroid was used in reactions. Reaction conditions were similar to glucuronidation with several modifications. In Phase I reactions, 20 μl of ovarian S9 were used with or without addition of cofactors (NAD+, NADH, NADP+, NADPH). The reaction time was two hours. Phase II reactions were performed with canine liver S9 with or without addition of cofactors and UDPGA. In Phase I+II reactions, after one-hour reaction time with ovarian S9, hepatic S9 were added with or without UDPGA and reactions were stopped after one hour. Samples without steroid and/or S9 were used as controls in the study. In mass spectrometric analysis (LC-qTOF-MS), pregnenolone, progesterone, 6α-, 6β-, 16α and 17α-hydroxyprogesterone were used as steroid standards. In addition, two diestrus phase urine samples were measured to compare the results with urinary metabolites.

### Synthesis of Reference Steroids

To identify the abundant urinary pregnanetriols, several steroids were chemically reduced, enzymatically glucuronidated and analyzed by mass spectrometry. All reference steroids were purchased from Steraloids unless otherwise noted. 5α-pregnane-3β,6α,20α-triol, 5α-pregnane-3β,6α,20β-triol and 5α-pregnane-3β,6α,20β-triol-6-O-β-glucuronide were ordered by custom synthesis by Toronto Research Chemicals.

### Reduction

For reference steroid synthesis, 16α-, 6α- and 6β-hydroxyprogesterone (OHP) were reduced by a method: 10% Pd/C in THF at 100 psi, stirring overnight. 5β-pregnane-3α,6α-diol-20-one, 5α-pregnane-3β,6α-diol-20-one and 5α-pregnane-3β,20β-diol-6-one reduced by a method described by Bournot et al. (1989) except for methanol was used instead of ethanol. In addition, 5α-pregnane-3β,20β-diol-6-one was reduced also selectively using a method:10% Pd/C in THE at 50 psi, stirring overnight. Pregnenolone (P5, 5-pregnene-3β-ol-20-one) was purchased from Sigma and reduced by a method described in Stappenbeck et al. (2012).

### Glucuronidation

The reduced steroids as well as 5β-pregnane-3α,6α-diol-20-one, 5α-pregnane-3β,6α,20α-triol and 5α-pregnane-3β,6α,20β-triol were glucuronidated enzymatically. 5β-pregnane-3α,6α,20α-triol-20-acetate and 5β-pregnane-3α,6α,20β-triol-20-acetate were glucuronidated by two different ways, before and after deacetylation. Steroids were deacylated by adding 10 M NaOH and vortexing vigorously (pH 10-11).

For analytical purposes, glucuronidation was performed in 100 μl volume as described in Example 1. Reduced pregnenolone was glucuronidated in larger amounts (3 μmol). Glucuronidation and purification was performed as in Gufford et al. (2015) with modifications. Steroid was dissolved in DMSO and added to 50 ml reaction tube. The conditions of the reactions were similar to smaller scale glucuronidation, but with addition of 0.5% BSA (w/V) and 25 μM alamethicin. The reaction was initiated by adding 1.25 mg of CLM and reaction tubes were incubated 20-24 hours. Ice-cold methanol (3 times reaction volume) was used as stopping solution. Glucuronidated steroid hormones were extracted from reaction solution by solid-phase extraction. The methanol was removed from the reaction solution by evaporating the solution to at least tenth of the original volume by the stream of nitrogen gas at +50° C. Silica-based C18 solid reversed-phase extraction column (Waters Sep-Pak C18 3 cc Vac Cartridge, 500 mg Sorbent per Cartridge, 55-105 μm (WAT020805)) was conditioned by draining 4 ml of methanol and followed by 3 ml of 10% methanol in ultrapure water. The concentrated glucuronide solution (1 to 2 milliliters) was diluted in 10% methanol in ultrapure water with 1:3 ratio and loaded to column. The column was rinsed with 3 ml of 10% methanol in ultrapure water. The flow-through liquid was disposed. The steroid glucuronides were eluted with 50% methanol in ultrapure water to glass tube. Free steroids and less polar glucuronides were eluted with 100% methanol. The eluates were inserted to heat block (+50° C.) and solvent was evaporated to dryness in nitrogen evaporator. The sample was dissolved in methanol to approximate 1 mg/ml concentration.

### Analysis of Reference Steroids by Mass Spectrometry

The commercial reference steroids were analyzed by mass spectrometry before anything was done on them. After chemical reductions, the steroids were analyzed by mass spectrometry both before and after glucuronidation. Steroids were diluted at 1-5 μM concentration in 30% acetonitrile and compared with urine samples. Freshly prepared samples were stored in the refrigerator until the analysis. The mass spectrometry analyses were performed using either Agilent Qualitative Analysis B.07.00 (or newer version) or Thermo Xcalibur Qual Browser 4.0 (or newer version). Analyses of the steroids were performed by negative and/or positive ionization and, in addition, the MS/MS spectrum was measured for some steroid molecules.

### Results

As described in Example 1, the glucuronidated synthetic pregnanetriols (PtGs, m/z 511) were compared with urine samples collected from diestrus phase of two bitches. When these urine samples were compared with each other, there were close-eluting pregnanetriol glucuronides at 6.6-6.7 minutes (PtG1 and PtG2).

### In Vitro Metabolism of Progesterone

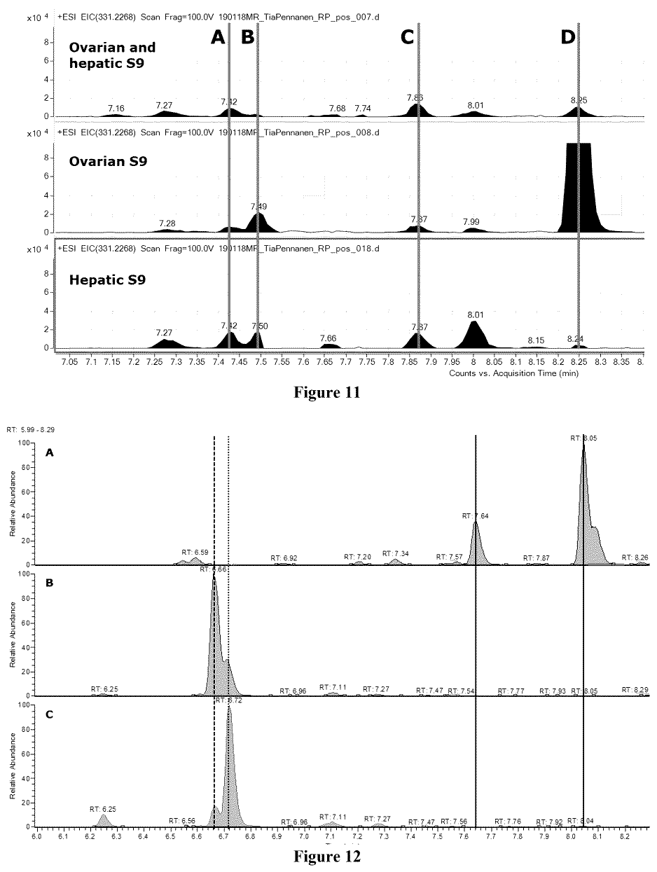

In vitro studies by canine ovarian and hepatic S9 showed that ovaries and liver have high 6α-, 6β-, 16α- and 17α-hydroxylase activity. When NADPH was present with progesterone, ovaries synthetized large amounts of 17α-OHP and smaller amounts of 6α-, 6β- and 16α-OHP. The 17α-OHP decreased significantly when hepatic S9 were added, which indicates that liver enzymes metabolize the 17α-OHP effectively (FIG. 11). Because the common urinary metabolite of 17α-hydroxyprogesterone in many mammals, 5β-pregnane,3α,17α,20α-triol, does not follow the 17α-OHP concentration in dog serum (Example 1), it is very likely that liver metabolized 17α-OHP is excreted to urine other compounds than pregnanetriols, as pregnanediol-ones or pregnanetetrols. Other hydroxyprogesterones did not decrease significantly after addition of hepatic S9, probably due to lower concentration. Unlike ovarian S9, hepatic enzymes produced only small amounts of 17α-OHP, or it was directly to metabolized further. Instead, higher amounts of 6α-, 6β- and 16α-hydroxyprogesterones were detected. In addition, several unidentified hydroxyprogesterones were produced, even though one of them (retention time 8.0 min), which is produced mainly in liver, was later identified as 21-OHP. High 6-hydroxylase activity in ovaries and liver reinforce the perception that the major urinary progesterone hormone metabolite (Example 1) was 5α-pregnane-3,6,20-triol.

### Identifying 5α-PtG by Reference Steroids

The 16α-hydroxyprogesterone (16α-OHP) was modified by chemical reduction and glucuronidated by CLM. In theory, all stereoisomers could be formed by reduction and result any 5-pregnane-3,16,20-triol-glucuronides. When chemically modified 16α-OHP was compared with diestrus phase urine samples, it could be clearly seen that majority of 16α-OH-based pregnanetriols are less polar than the urinary PtGs (FIG. 12). The urinary PtGs are not formed in reduction and glucuronidation of 16α-hydroxyprogesterone.

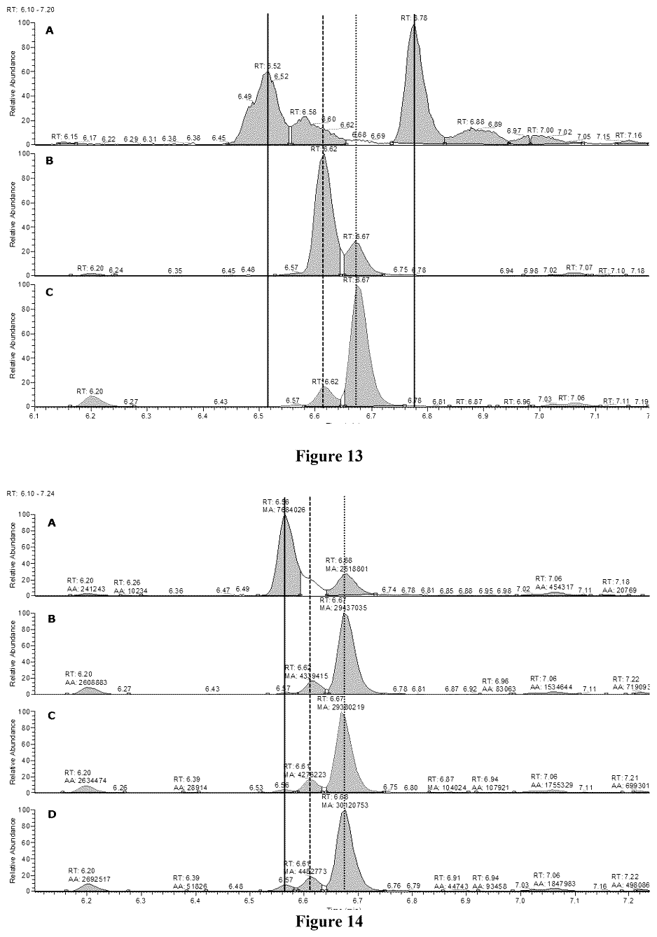

The reduction and glucuronidation of 6β-hydroxyprogesterone (6β-OHP) provided several PtGs (FIG. 13). Corresponding molecules of the synthesized 6β-pregnanetriol glucuronides are not found in dog urine. The reduction and glucuronidation of 6α-hydroxyprogesterone (6α-OHP) provide few PtGs, in which the most abundant one was detected at 6.56 minutes, which cannot be found from urine samples (FIG. 14). However, the shoulder at a left-side peak at 6.56 minutes and the peak detected at 6.67 min indicate that urinary PtGs are 6α-pregnanetriol isomers.

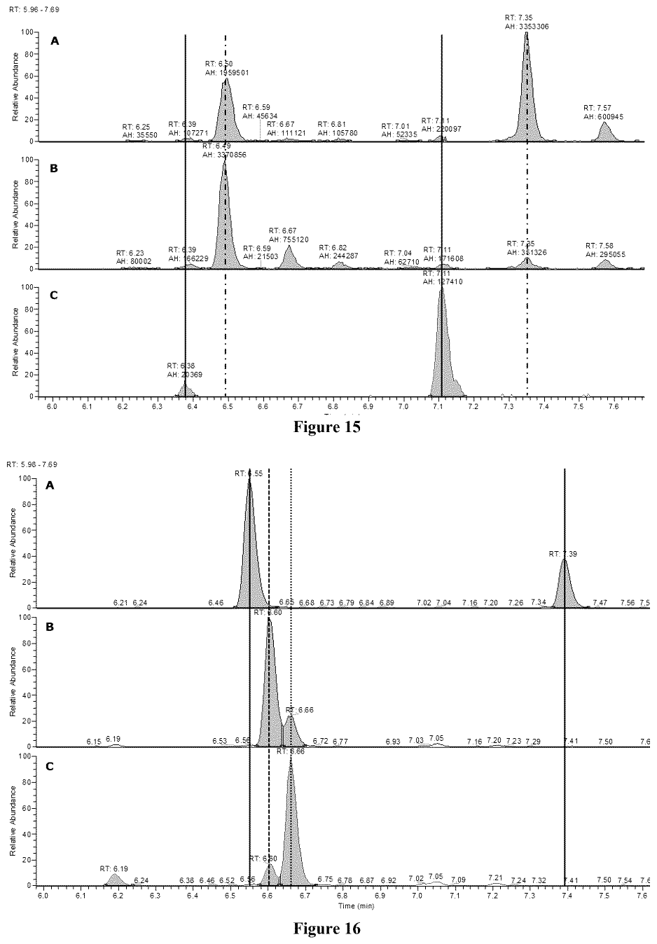

Urine samples were compared with glucuronidated 5β-pregnane-3α,6α-diol-20-one (m/z 509). 5β-pregnane-3α,6α-diol-20-one is a known progesterone metabolite in a sow (Jones and Erb, 1968). Urinary samples show two abundant pregnanediol-one-structured metabolites, which do not match with the reference steroid (FIG. 15).

The 5β-pregnane-3α,6α,20α/β-triols were synthetized by chemical reduction. The reduction of 5β-pregnane-3α,6α-diol-20-one provides two reference steroids: 5β-pregnane-3α,6α,20α-triol and 5β-pregnane-3α,6α,20β-triol. The synthesis products were glucuronidated and compared with diestrus urine sample of a dog. In addition, 5β-pregnane-3α,6α,20α-triol-20-acetate and 5β-pregnane-3α,6α,20β-triol-20-acetate were glucuronidated before and after deacetylation.

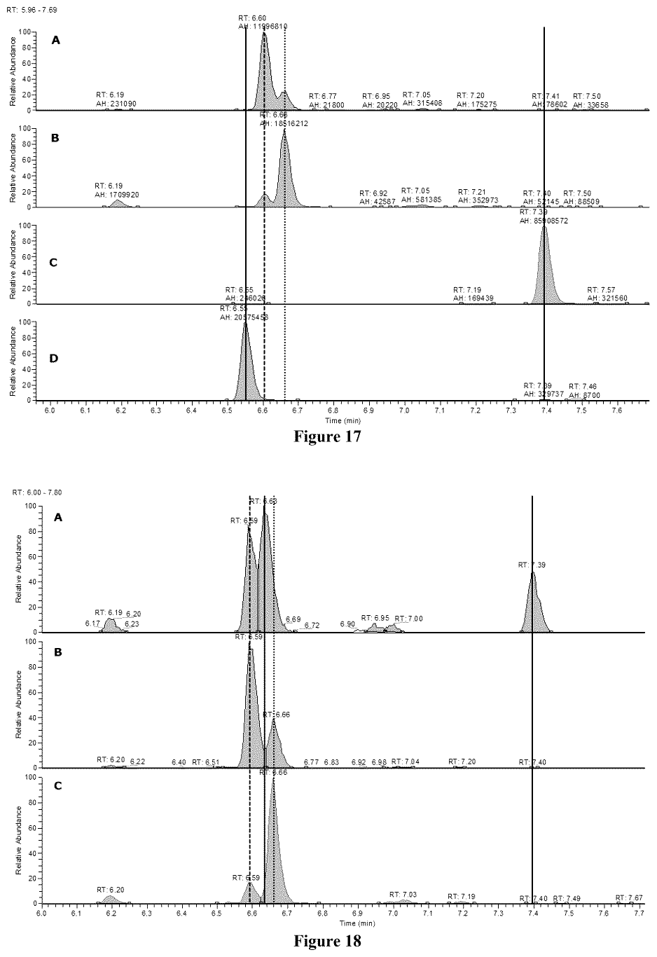

The reduction and glucuronidation of 5β-pregnane-3α,6α-diol-20-one resulted in two abundant steroid glucuronides at retention time 6.55 minutes and 7.39 minutes (FIG. 16). The difference between the synthetized pregnanetriol glucuronide and the abundant metabolites were 0.05 and 0.11 minutes. Only traces of the 5β-pregnane-3α,6α,20α/β-triol-glucuronides were found in dog urine. The area of the corresponding peak was 0.9-1.1% from the abundant urinary PtG metabolite. 5β-pregnane-3α,6α,20α-triol has been measured from the urine of Asian elephant, rabbit and human newborns (Anderson et al., 1974; Niemuller et al., 1993; Senciall et al., 1990). When 50-pregnane-3α,6α,20α/β-triol-20-acetates were deacetylated and glucuronidated, it was seen that CLM form only one glucuronide, even though the pregnanetriol has three OH-groups available (FIG. 17). The result indicates that either the glucuronidation of progesterone metabolites is highly dependent on the configurations of the hydroxyl groups, or we can't mimic exactly the natural reactions in vitro with CLM.

Reduction of 5α-pregnane-3β,6α-diol-20-one could produce 5α-pregnane-3β,6α,20α/β-triols, which can be glucuronidated from hydroxyl groups in C3, C6 and C20, providing 6 steroid references in total. The analysis of reduced and glucuronidated steroids revealed 5-6 steroids, in which one retention time was same with PtG1 (FIG. 18). The left side of the split peak matched with urinary PtG1 (RT 6.59 minutes), whereas the right side was detected slightly earlier than PtG2 (6.63 minutes vs 6.66 minutes).

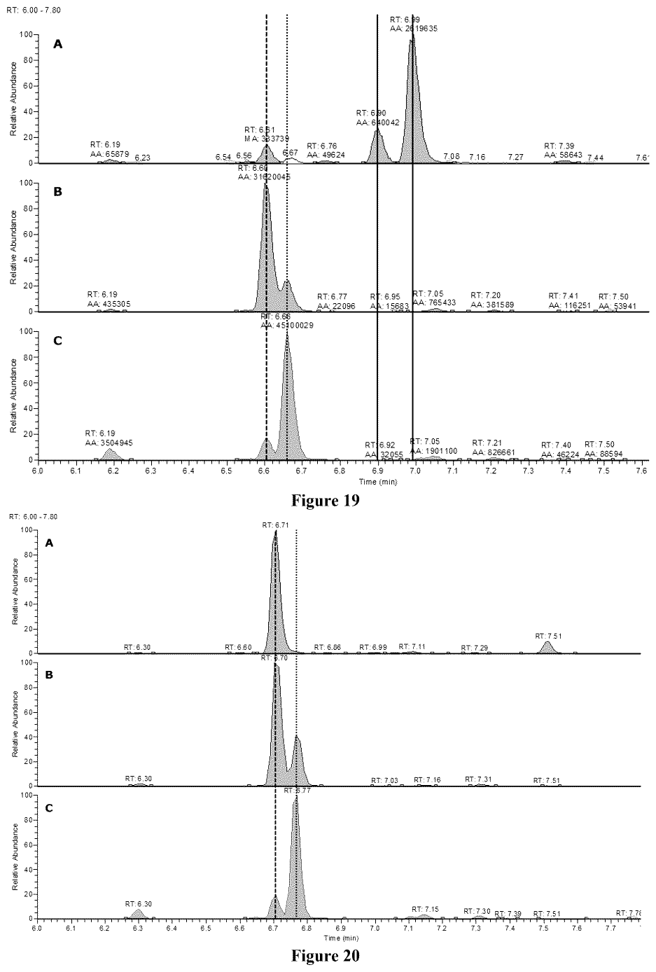

Reduction and glucuronidation of 5α-pregnane-3β,20β-diol-6-one produced three major products (FIG. 19). One of the peaks was concurrent with PtG1, indicating that PtG1 was 5α-pregnane-3β,6α/β,20β-triol. When previous results of reduced and glucuronidated 6α-OHP and 5α-pregnane-3β,6α-diol-20-one were taken into account (see FIGS. 14 and 18), it is highly probable that the PtG1 was 5α-pregnane-3β,6α,20β-triol-glucuronide.

5α-pregnane-3,6,20-triols were synthetized from pregnenolone (P5, 5-pregnene-3β-ol-20-one) by reduction and glucuronidated by CLM. The mixture of glucuronidated and free steroids was purified by solid-phase extraction (SPE) with 10, 50 and 100% methanol. Two major PtGs were formed in steroid synthesis, which could be separated by SPE. Mass spectrometric analysis showed that retention times of PtGs in 50% methanol fraction and the abundant urinary metabolite are identical (FIG. 20). According to the MS2 spectrum, the synthetic PtG and urine metabolite were similar (Table 4). According to the synthesis route (Stappenbeck et al., 2012) and all the results achieved, PtG1 metabolite was identified as 5α-pregnane-3β,6α,20β-triol glucuronide.

TABLE 4
Spectrum lists of m/z 511 at retention time 6.7
minutes of synthetic 5α-pregnane-3,6,20-
triol glucuronide, and urine sample.

| 5α-pregnane-3,6,20-triol- | | Urine sample, diestrus | |
|---|---|---|---|
| glucuronide RT 6.7 min | | phase RT 6.7 min | |
| m/z | Relative | m/z | Relative |
| 57.03 | 3.88 | 57.03 | 5.1 |
| 59.01 | 1.81 | 59.01 | 2.18 |
| 71.01 | 3.9 | 71.01 | 4.81 |
| 72.99 | 1.67 | 72.99 | 2.35 |
| 73.03 | 1.66 | 75.01 | 33.45 |
| 75.01 | 37.08 | 83.01 | 1.52 |
| 83.01 | 1.51 | 85.03 | 30.78 |
| 85.03 | 32.22 | 87.01 | 1.99 |
| 87.01 | 1.26 | 95.01 | 2.28 |
| 95.01 | 3.56 | 99.01 | 2.54 |
| 99.01 | 2.06 | 113.02 | 21.89 |
| 113.02 | 21.14 | 115 | 1.16 |
| 149.68 | 1.27 | 116.01 | 1.47 |
| 157.01 | 3.11 | 117.02 | 0.81 |
| 210.7 | 1.3 | 129.02 | 1.59 |
| 305.9 | 1.38 | 157.01 | 2.02 |
| 311.22 | 1.36 | 175.02 | 2.22 |
| 344.56 | 1.32 | 393.27 | 0.72 |
| 493.28 | 1.74 | 493.28 | 1.35 |
| 511.29 | 100 | 511.29 | 100 |

In PtG identification, the different PtG molecules obtained in reductions or contained in urine could not be separated for NMR analysis. 6α-OHP, 6β-OHP, 5β-pregnan-3,6,20-trione, 5α-pregnan-3β,20β-diol-6-one were chemically reduced and the resulting pregnanetriol mixtures and a hydrolyzed urine sample were fractionated by preparative HPLC coupled with MS detector. Ionization was performed with positive electrospray ionization. Separation was achieved on a Phenomenex Gemini C18 column (150 mm×21.2 mm, 5 m) using an isocratic mobile phase system composed of methanol-ammonium formate (1 mM)—formic acid (50:40:0.1, V/V/V) at a flow rate of 1 ml/min and fractions were collected 1 minute time intervals.

The fractions were inserted to heat block (+50° C.) and solvent was evaporated to dryness in nitrogen evaporator. The fractions were dissolved in 3000 acetonitrile and analyzed by mass spectrometer (LC-QTOF-MS, m/z 354 [M+NH4]+ and m/z 301 [M−2H2O+H]+) parallel with hydrolyzed urine samples (from bitches in the diestrus phase, untreated) and original pregnanetriol mixtures (from reduced starting molecules). The results showed which of the reduced molecules formed the same pregnanetriol molecules found in dog urine.

Next, the fractions were enzymatically glucuronidated as above and subjected to mass spectrometric analysis (LC-QTOF-MS, m/z 511 [M-H]−) in parallel with glucuronidated original pregnanetriol mixtures (from reduced starting molecules) and untreated urine samples (from bitches in the diestrus phase). The results showed the fractions containing the PtG1 and the PtG2 and from which starting molecule they were obtained.

Based on the results, 5α-pregnane-3β,20β-diol-6-one was chosen as the starting molecule, which reduction products were fractionated in several batches. Fractions were analyzed with the LC-MS and fractions containing the pregnanetriols with same retention time were pooled. The pregnanetriol pools were glucuronidated and analyzed with the LC-MS before and after the glucuronidation. Any of the pools did not contain the PtG2. NMR analysis of the pool containing the PtG1 molecule indicated that the glucuronide was attached to the 6-carbon of pregnanetriol.

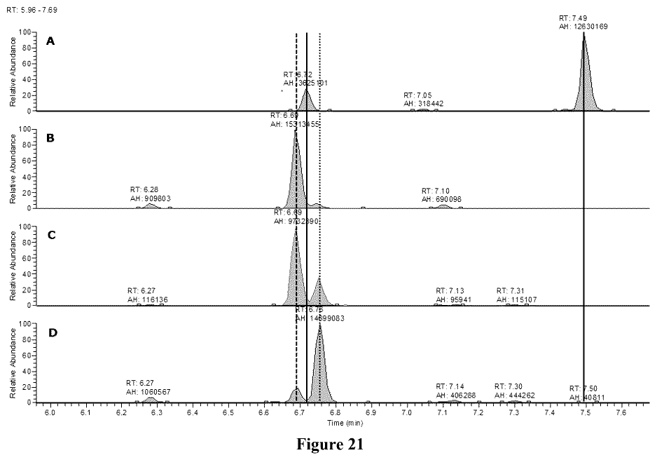

Glucuronidated custom-made molecules 5α-pregnane-3β,6α,20α-triol and 5α-pregnane-3β,6α,20β-triol were compared with urine samples of two bitches. The results showed that the major peak of 5α-pregnane-3β,6α,20β-triol glucuronides and PtG1 matched (FIG. 21). NMR analysis of 5α-pregnane-3β,6α,20β-triol glucuronide confirmed that the glucuronide was attached to the 6-carbon of pregnanetriol.

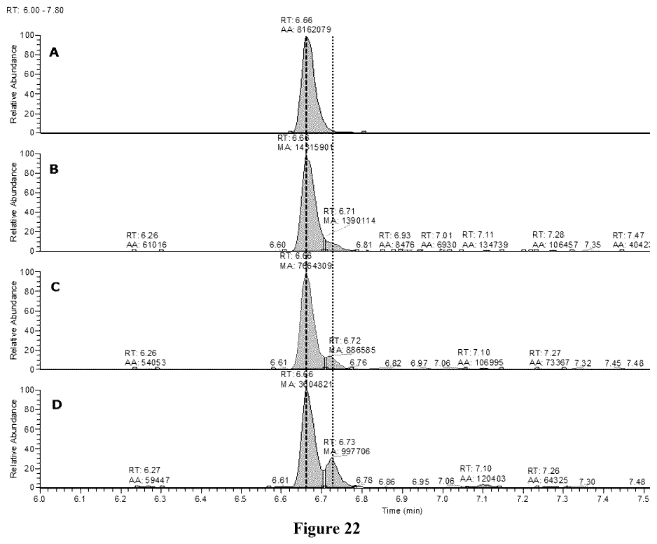

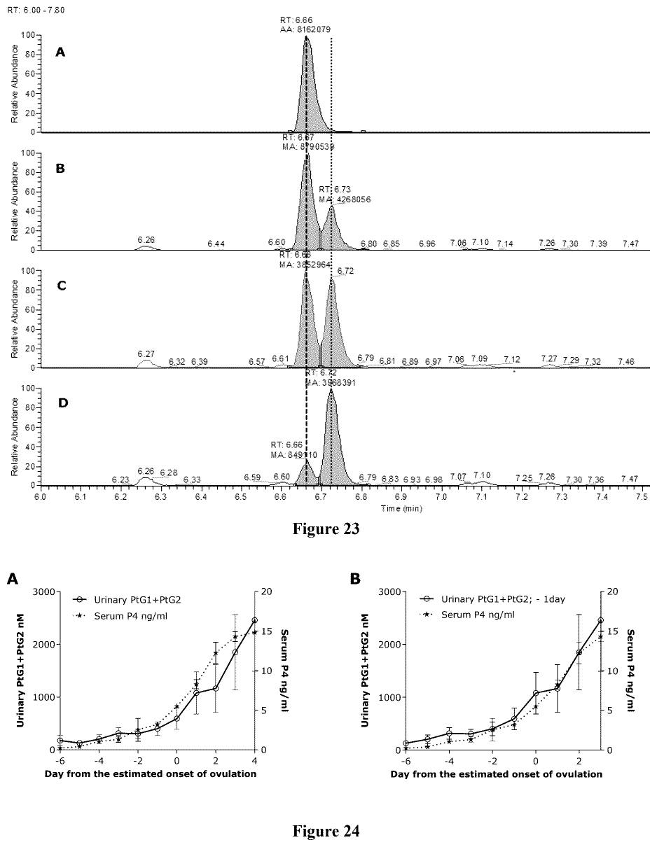

Custom-made 5α-pregnane-3β,6α,20β-triol-6-O-β-glucuronide was compared with urine samples of two bitches with and without addition of the reference steroid (FIGS. 22-23). The results showed that the reference and PtG1 are detected at the same retention time. The spiked samples showed linear increase in area of the peak. The PtG1 was confirmed to be 5α-pregnane-3β,6α,20β-triol-6-O-β-D-glucuronide. Due to similar excretion profile to PtG1, it is highly probable that PtG2 is a stereoisomer of PtG1.

### Example 4

Quantitative Measurement of 5α-Pregnane-3,6,20-Triol (5α-PtG) with LC-HRMS

New urinary biomarker for circulatory progesterone was found to be 5α-pregnane-3β,6α,20β-triol-6-O-β-D-glucuronide. LC-HRMS and enzyme immunoassay methods were developed to measure the quantity of the urinary progestogen metabolite in a dog.

Aliquoted urine sample series of eight bitches and individual samples from 89 bitches in estrus or diestrus phase were thawed to room temperature, vigorously vortexed and centrifuged 5 minutes at 13 400 rpm. Samples were diluted 1:3 in ultrapure water and mixed with 200 nM diclofenac in acetonitrile 1:1 (diluted water:ACN). The urine sample solution was filtered through 96 well plate by centrifugation (2300 rpm, 5 min) to 1 ml deep well plates and sealed by silicone well cap. Standard dilution series (1-1000 nM) of 5α-pregnane-3β,6α,20β-triol-6-O-β-D-glucuronide was prepared in 30% ACN with 100 nM diclofenac. Standard urine samples were spiked with addition of 5α-pregnane-3β,6α,20β-triol-6-O-β-D-glucuronide. 30% ACN was used as blank control and pooled urine sample as a quality control. The urine samples were analyzed by Thermo Scientific Q Exactive Quadrupole-Orbitrap high resolution MS/MS system with Thermo Scientific Vanquish Binary Flex Binary 1000 bar UHPLC system; method similar to method used in non-targeted analysis. Two close-eluting isomers (m/z 511 [M-H]−) are found from extracted ion chromatograms at retention time of 6.7 minutes. Both metabolites were used in analysis and oestrus monitoring. Also, semi-quantitative analysis of PdG was performed by comparing peak areas in extracted ion chromatograms of PdG (m/z 495) and 5α-PtG (m/z 511).

Quantitative data analysis was performed with TraceFinder 5.0 application. The results were normalized by dividing the integrated area of the 5α-PtG with the area of the diclofenac and calculated with the equation of the standard curve (nM).

### Results

Mass spectrometric analysis by Thermo Scientific Q Exactive Quadrupole-Orbitrap high resolution MS/MS revealed that progesterone is metabolized as several different metabolites, including overlapping, two close-eluting isomers of pregnanetriol glucuronide, in which the first-eluting peak (PtG1) corresponded with 5α-PtG. The increase of PtG1 was linear when 5α-PtG was added to urine samples, confirming that the steroids are identical. Due to close-elution and similar profiles, metabolites are assumed to be stereoisomers of each other, and referred as general PtG in these results.

The excretion ratio of these metabolites varies between individuals, but all bitches excrete both PtG1 (5α-PtG) and PtG2, and both PtGs follow the profile of serum progesterone. These metabolites can be used as oestrus monitoring as individual metabolites or by summing up the metabolites. The mass spectrometric measurement with lower resolution showed only one peak for both PtGs. Thus, these results are shown as a sum of PtG1 and PtG2.

According to quantitative measurements, the average concentration of urinary 5α-PtG (the sum of PtG1 and PtG2) increases to 2.5 μM concentration in four days after ovulation and show similar profile than serum progesterone (serum progesterone analysis was measured and informed by dog owners). Results suggest that the progesterone changes can be detected at latest at the next day from the urine sample (r²=0.9628) (FIG. 24). When the increase (%) of PtGs was compared to average concentration before estimated LH peak (−2 days from estimated onset of ovulation), 5α-PtG concentration increases over 100% from the beginning of LH peak to estimated onset of ovulation. During the six-day period (from −2 to +4 days), concentration increased 800%.

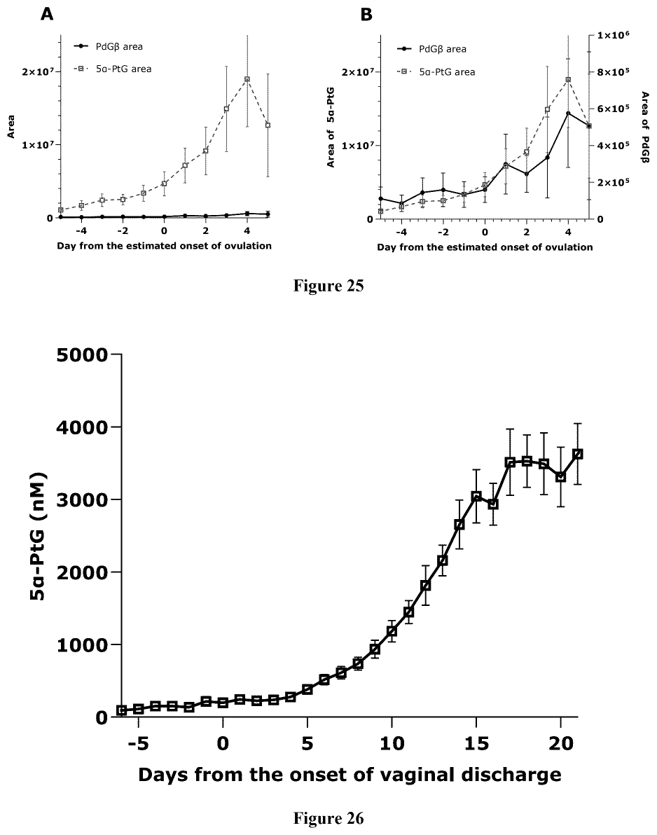

The superiority of this new biomarker, 5α-PtG, was demonstrated by comparing it semi-quantitatively with the 20β-stereoisomer of the most common progesterone metabolite, PdG (FIG. 25A). In dogs, PdG is metabolized mostly in 20β-form (PdGβ). According to these results, the excretion of the PdG 20β-form resembles the PtG excretion (FIG. 25B). However, due to low concentrations it is not suitable biomarker for progesterone excretion. In average in eight bitches, the area of PdGβ (extracted ion chromatogram) was only 4.3% from the area of 5α-PtG. The amount of PdGβ varied between individuals. The average percentage (area of PdG/5α-PtG*100%) in individuals varied between 0.3 to 17.3.

### Example 5

### Quantitative Measurement of 5α-PtG by Enzyme Immunoassay

Imject™ mariculture keyhole limpet hemocyanin (mcKLH) in PBS, Imject™ bovine serum albumin (BSA) in PBS and Imject® cBSA Immune Modulator in PBS (cBSA) were used as carrier proteins in immunization, antibody screening and urinary measurements. Hapten-protein conjugation was performed with EDC (1-Ethyl-3-(3-dimethylaminopropyl)-carbodiimide hydrochloride) and sulfo-NHS (N-Hydroxysulfosuccinimide sodium salt). Both EDC and sulfo-NHS were dissolved in MES buffer (0.1 M 2-(N-morpholino)-ethanesulfonic acid (0.9% NaCl, pH 4.6) right before use. Antigen (5α-PtG, 5α-pregnane-3β,6α,20β-triol-6-O-β-D-glucuronide) was dissolved in DMSO to 10 mg/ml (19.5 mM) concentration.

In conjugations, 500 and 50 molar excesses of the 5α-PtG were used in KLH and (c)BSA conjugations, respectively. The molarity of EDC was 1.5 times and sulfo-NHS 1.2 times to 5α-PtG molarity. The freshly dissolved sulfo-NHS was added to glass vial containing the 5α-PtG and vortexed thoroughly. The activation of the steroid was initiated by addition of fresh EDC solution and vigorous vortexing. The reaction solution, consisting of DMSO and MES buffer in 1:1 ratio, was incubated at room temperature in plate shaker (300 rpm) for an hour. After activation, the reaction solution was slowly added to protein solution, vortexed gently and incubated at plate shaker (300 rpm) for two hours. The protein conjugates were purified by centrifugal filtration (30 k MWCO for KLH and 10 k MWCO for BSA conjugates). The unconjugated steroids, conjugation buffer and DMSO were removed by centrifugation (4000 rpm, 10-20 minutes) and protein concentrates were washed three times with 5 ml of PBS (pH 7.4). The conjugate was diluted in PBS in 1-5 mg/ml concentration and stored in refrigerator until use.

Monoclonal mouse 5α-PtG antibody was raised against the 5α-PtG-KLH by Thermo Fisher Scientific (Rockford, TL, USA). The antibodies were screened against the 5α-PtG-BSA. Cross-reactivities for the antibody were: 100% 5α-pregnane-3β,6α,20β-triol glucuronide, 25.4% 5α-pregnane-3β,6α,20β-triol, 0.5% 5β-pregnane-3α,20β-diol and 0.5% 5α-pregnane-3β,6α-diol-20-one. No cross-reactivities were discovered against 5α-pregnane-3β,6α,20α-triol, 5β-pregnane-3α,20α-diol-3-glucuronide, 5β-pregnane-3α,20α-diol, 5β-pregnane-3α,17α,20α-triol, 5β-pregnane-3α,17α,20β-triol, 5α-pregnane-3β,17α,20β-triol or progesterone.

The 96 well polystyrene, certified MaxiSorp® plates were coated with antigen (5α-PtG-cBSA) by adding 100 μl of 0.25 μg/ml antigen in coating buffer (0.01 M sodium bicarbonate, pH 9.5) in each well. The plates were covered with plastic sticker to prevent evaporation, protected from light and incubated at room temperature over night or several nights at +4° C. The wells were emptied and washed once with 250 μl general washing buffer (e.g. PBS with 0.05% Tween 20 (PBST)), filled with 200 μl blocking buffer (5% sucrose and 0.5% BSA in PBST (w/V)), covered and incubated at +37° C. for a half an hour. The plates were poured empty and dried for three hours at +37° C. Dried plates were covered with plastic sticker, packed in re-sealable plastic bags with silica beads and stored at +4° C. until use.

The hormone assays were performed from the individual samples collected in different oestrus cycle phases and from the urine sample series from 60 bitches. The 5α-PtG-cBSA coated plates were washed once with 250 μl of PBST. The urine samples were diluted 1:20 in assay buffer (0.2% BSA (w/V) in PBS with 0.01% Triton-X) and added in sample wells (50 μl). A dilution series of standard was prepared by diluting the 5α-PtG in assay buffer and added to standard wells (50 μl; 1200-1.6 nM). Assay buffer without additives was added to two wells to determine the maximum binding capacity (B0) and 1900 nM 5α-PtG control was diluted 1:20 in assay buffer and used as a recovery control in all assays. All samples and standards were measured as two replicates. The stock dilution of monoclonal mouse 5α-PtG antibody (1:100) was further diluted 1:100 in assay buffer and added 50 μl in each well. The plate was covered and incubated at +37° C. for half an hour, followed by washing the plate three times. Horseradish peroxidase labeled secondary antibody (goat anti-mouse IgG HRP) was diluted in 1:20 000 in 0.5% BSA (w/V) in PBST, added 100 μl per well and incubated half an hour at +37° C. After washing the plate three times with PBST, TMB in DMSO solution (0.5% 3,3′,5,5′-tetramethylbenzidine in dimethylsulfoxide (w/V; TCIAT1023 Tokyo Chemical Industry)) was diluted 1:50 in substrate buffer (0.1 M sodium acetate trihydrate, 1.5 mM citric acid monohydrate, 0.005% hydrogen peroxide (30%)), added in each well (100 μl) and incubated at room temperature 30 minutes. The reaction was stopped by adding 50 μl 1 M sulfuric acid to each well. The absorbance was measured at 450 nm by microplate reader (Hidex Sense).

The results were calculated by four parameter logistic curve of standard dilution series. The 4PL curve fitting is made by AssayFit v 1.4.1 and results are calculated by equation:

$$Concentration ( nM ) = dilution factor * {c ( \frac{a - d}{( y - d )} - 1 )}^{\frac{1}{b}} ,$$

where a is the minimum and d is the maximum values that can be obtained, c is the point of inflection, y is the absorbance at 450 nm of the sample, and b is the Hill's slope (steepness of the curve) of the curve. The sample concentration (nM) was calculated from two parallel wells (±SD) and normalized by dividing the result by creatinine concentration (pmol/pmol Cr). The average 5α-PtG profile was determined as an average concentration of 5α-PtG of each day (±SEM). Day 0 was considered as the day of the onset of vaginal discharge. If serum progesterone values were known, ovulation was estimated to occur when serum progesterone was 5-6 ng/ml or two days after 2 ng/ml of progesterone (estimated LH peak).

### Results

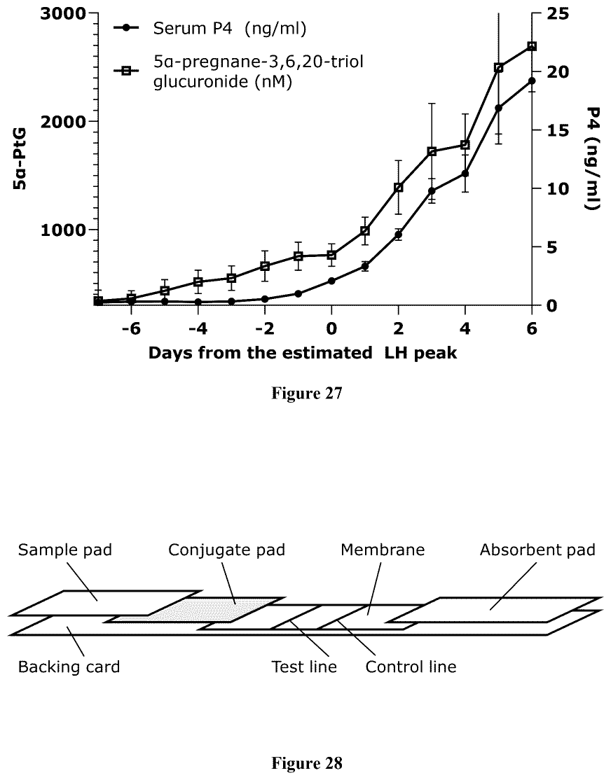

Progestogen metabolite concentrations were quantified by enzyme immunoassay (EIA). In total of 60 urine sample series were measured by the 5α-PtG EIA. According to the results, 5α-PtG concentration is low during anoestrus and proestrus, and average concentration starts to increase approximately one week after the onset of vaginal discharge and reach the maximum concentration under three-week period (FIG. 26). When urinary 5α-PtG concentrations were compared with the serum progesterone profile (n=27 bitches, FIG. 27), correlation coefficient was 0.988.

The results of 5α-PtG measurements are remarkable. This new, previously unknown metabolite can be used as a biomarker for serum progesterone, which has been used to monitor the phase of the oestrus cycle in a dog in practise and in research. New urinary biomarker provides non-invasive methods to monitor oestrus cycle phase in a bitch. Results are encouraging and methods are easily modified to consumable forms.

### Example 6

### Quantitative Measurement of 5α-PtG by Capture Enzyme Immunoassay

Urinary 5α-PtG was measured by enzyme immunoassay with capturing antibody. The reagents, buffers and analyzing methods were as in Example 5 if not noted otherwise. This assay provides an alternative method to antigen-based EIA. For horseradish peroxidase (HRP) conjugation, 5α-PtG was conjugated to HRP (Thermo Scientific, 31490) with similar process to 5α-PtG-BSA conjugation (Example 5), but the molarity of 5α-PtG was eightfold to HRP molarity. The 96 well polystyrene plates (Thermo Scientific) were coated by adding 100 μl of 1 μg/ml anti-mouse IgG (GtxMu-003-D, goat anti-mouse IgG (H&L), ImmunoReagents) in coating buffer in each well. The plates were covered with plastic sticker to prevent evaporation, protected from light, and incubated at room temperature over night or several nights at +4° C. The wells were emptied and washed three times with 250 μl general washing buffer right before use.

The stock dilution of monoclonal mouse 5α-PtG antibody was diluted 1:20 000 in assay buffer and added 100 μl in each well. The plates were covered and incubated at +37° C. for half an hour, followed by washing the plate three times. The urine samples were diluted 1:20 in assay buffer and a dilution series of standard was prepared by diluting the 5α-PtG in assay buffer. Horseradish peroxidase labeled 5α-PtG was diluted in 1:10 000 in 0.5% BSA (w/V) in PBST and added to diluted urine samples and standards (1:1). 100 μl of the mixture of urine sample/or standard and HRP labeled 5α-PtG was added to well plates and plates were incubated for half an hour at +37° C. After washing the plate three times with PBST, TMB in DMSO solution was diluted 1:50 in substrate buffer, added in each well (100 μl) and incubated at room temperature for 30 minutes. The reaction was stopped by adding 50 μl 1 M sulfuric acid to each well. The absorbance was measured at 450 nm by microplate reader.

### Example 7

### Quantitative Measurement of 5α-PtG by Alternative Capture Enzyme Immunoassay

This immunoassay is based on the method used in capture EIA in Example 6. The reagents, buffers and analyzing methods were as in Example 5 and 6 if not noted otherwise. This assay is a simplified version of capture EIA.

The 96 well polystyrene plates (Thermo Scientific) were coated by adding 100 μl of 0.2 μg/ml monoclonal 5α-PtG antibody IgG in coating buffer in each well. The plates were covered with plastic sticker to prevent evaporation, protected from light, and incubated at room temperature over night or several nights at +4° C. The wells were emptied and washed three times with 250 μl general washing buffer right before use.

The urine samples were diluted 1:20 in assay buffer and a dilution series of standard was prepared by diluting the 5α-PtG in assay buffer. Horseradish peroxidase labeled 5α-PtG was diluted in 1:10 000 in 0.5% BSA (w/V) in PBST and added to diluted urine samples and standards (1:1). 100 μl of the mixture of urine sample/or standard and HRP labeled 5α-PtG was added to well plates and plates were incubated for an hour at room temperature. After washing the plate three times with PBST, TMB in DMSO solution was diluted 1:50 in substrate buffer, added in each well (100 μl) and incubated at room temperature for 30 minutes. The reaction was stopped by adding 50 μl 1 M sulfuric acid to each well. The absorbance was measured at 450 nm by microplate reader.

### Example 8

### Qualitative Analysis of 5α-Pregnanetriol Glucuronides by Immunoaffinity Chromatography and LC-HRMS.

The antibody cross-reacting steroids were analyzed by immunoaffinity chromatography. Cyanogen bromide (CNBr)—activated Sepharose™ 4B (1 g, Cytiva 17-0430-01, Uppsala, Sweden) was suspended and washed multiple times with 1 mM HCl (200 ml, Sigma-Aldrich 30721). The sepharose medium was transferred to PD10 column and rinsed five times with coupling buffer (0.1 M NaHCO₃, 0.5 M NaCl, pH 8.4). Three milligrams of 5α-PtG antibody was diluted 1:5 in coupling buffer and added to the column. The sealed column was incubated in gentle shaking at room temperature for four hours. The column was rinsed two times with 5 ml of coupling buffer and blocked by adding 10 ml of blocking buffer (0.1 M Tris-HCl, pH 8) and incubating the column two hours at room temperature. The column was washed by three cycles of alternating pH. At first, the column was rinsed by 5 ml of acid buffer (0.1 M sodium acetate, 0.5 M NaCl, pH 4) and followed by 5 ml of the alkaline buffer (0.1 M Tris-HCl, 0.5 M NaCl, pH 8). Prepared column was rinsed five times with 5 ml of PBS, followed by 10 ml of 20% ethanol. After draining few milliliters of 20% ethanol, the column was sealed and stored at +4° C.

Before use, the a-5α-PtG-column was rinsed three times with 5 ml of PBS. Urine sample (3 ml) was diluted with 2 ml of PBS and added to the sealed column. The column was incubated 30 minutes at room temperature with gentle shaking. After incubation, the sample was drained and the eluate was collected for 5α-PtG EIA testing. Column was rinsed four times with 5 ml of PBS and cross-reactive steroids were eluted by 80% methanol to separate fractions (in total of 12 fractions, 1 ml of each). After elution, the column was rinsed three times with 5 ml of PBS and stored in 20% ethanol as before.

Fractions were dried by centrifugal evaporator (50° C.) and suspended in 5% methanol (100 μl). The fractions were diluted 1:50 to sample buffer and tested by 5α-PtG EIA as described in Example 5. The fractions that inhibited most in EIA were transferred to glass vials and analyzed by liquid chromatography-mass spectrometry. Non-targeted analysis of pregnanetriol glucuronides was carried by Thermo Scientific Vanquish Binary Flex Binary 1000 bar UHPLC system as described in Example 1.

According to the results, urinary 5α-PtG can be extracted from urine samples by immunoaffinity chromatography and detected by LC-IRMS (EIC m/z 511). No other significant cross-reacting pregnanetriol glucuronides was detected.

### Example 9

### Analysis of Hydrolyzed Urinary Steroids

The urinary steroid glucuronides were hydrolyzed before analysis. Centrifuged urine samples were diluted 1:3 in 75 mM KH₂PO₄ (pH 6.8) and 250-1250 units of type IX-A β-glucuronidase enzyme (Sigma G7396) was added. The hydrolysis was performed at +37° C. for 20 hours and unconjugated steroids are extracted by SPE. The SPE method is similar Example 1 (Sample preparation by solid-phase extraction), but unconjugated steroids were eluted by 100% methanol. Hydrolyzed urine samples could be used in oestrus detection by LC-MS analysis as described in Examples 1 and 2. Unconjugated pregnanetriols are detected by positive ionization at m/z 301 [M−2H2O+H]+ and m/z 354 [M+NH4]+. Unconjugated pregnanetriols can be measured by 5α-PtG EIA as in Example 5.

### Example 10

### Urinary Rapid Test for 5α-PtG Detection

Urinary 5α-PtG concentration differences between oestrus cycle phases in a dog were tested by a lateral flow chromatography. This rapid test provides a quick and cost-effective method to discover the urinary progestogen levels in a dog without the need for specialized equipment or laboratory facilities.

Lateral flow immunochromatographic test (FIG. 28) includes sample pad, conjugate pad, nitrocellulose membrane and absorbent pad attached to the backing card and cut into 4 mm wide test strips. The 5α-PtG antibody conjugated to the gold particle is dried on the conjugate pad. The 5α-PtG-BSA conjugate is immobilized to the nitrocellulose membrane as a test line and anti-mouse IgG antibody as a control line.

In the rapid test, the sample is placed on a sample pad and the sample flows through the test strip by capillary forces. On the conjugate pad, the 5α-PtG molecule contained in the sample binds to the 5α-PtG antibody conjugated to gold nanoparticle, and the formed complex moves to the membrane. If only a small amount of the 5α-PtG was present in the sample, the free antibody-gold nanoparticles will flow through the membrane and bind to the 5α-PtG-BSA in the test line, producing a visible signal. When the 5α-PtG molecules in the sample bind to the flowing antibody-gold nanoparticles, the antibodies do not interact with the 5α-PtG-BSA conjugate, and no visible test line is formed. Remained antibody-gold particles will bind to the anti-mouse IgG in the control line, producing a visible control line confirming that the fluid has successfully passed through the sample pad and test line to the control line. As the intensity of the test line is dependent on the amount of antigen present in the sample, the estimation of the oestrus cycle phase can be accomplished by simple visual evaluation. The excess sample is eventually absorbed into the absorbent pad, which also contributes to the capillary forces on the test strip.

The urine samples were tested without any preliminary preparation. In the pilot study, 80 μl of urine sample was applied to the sample well. The intensity of the test line was estimated, and the presence of the control line was confirmed after twenty minutes of sample application. The anoestrus phase urine sample with only a small amount of 5α-PtG provided a test line with the similar intensity to the control line. The diestrus phase urine with high concentration of 5α-PtG provided a hardly visible test line. When the intensity of the test line of the anestrus phase sample was determined as 100%, the intensity of the test line in proestrus phase was approximately 90%, in oestrus phase 50% and diestrus phase 10%.

### REFERENCES

Anderson, Chambaz, Defaye, Madani, Baillie, Brooks, 1974. Steroids in the human newborn; lipophilic gel separation and gas phase analysis. J. Chromatogr. Sci. 12, 636-641.
Beesley, Buglio, 2000. Quantitative Chromatographic Analysis, Quantitative Chromatographic Analysis.
Concannon, 2009. Endocrinologic control of normal canine ovarian function. Reprod. Domest. Anim. 44, 3-15.
Concannon, 2011. Reproductive cycles of the domestic bitch. Anim. Reprod. Sci. 124, 200-210.
Concannon, Castracane, Temple, Montanez, 2009. Endocrine control of ovarian function in dogs and other carnivores 6, 172-193.
England, Allen, 1989. Crystallization patterns in anterior vaginal fluid from bitches in oestrus. J. Reprod. Fertil. 86, 335-339.
Eriksson, Gustafsson, 1970. Excretion of Steroid Hormones in Adults: Steroids in Urine from a Pregnant Woman. Eur. J. Biochem. 16, 268-270.
Fennessey, Pike, Gonzalez-Aller, Horwitz, 1986. Progesterone metabolism in T47Dco human breast cancer cells-I. 5α-pregnan-3β,6α-diol-20-one is the secreted product. J. Steroid Biochem. 25, 641-648.
Fontbonne, Malandain, 2006. Ovarian ultrasonography and follow-up of estrus in the bitch and queen. Waltham Focus 16, 22-29.
Frank, Mullins, Rohrbach, 2010. Variability of estradiol concentration in normal dogs. Vet. Dermatol. 21, 490-493.
Günzel, Koivisto, Fougner, 1986. Electrical resistance of vaginal secretion in the bitch. Theriogenology 25, 559-570.
Häkkinen, Heinosalo, Saarinen, Linnanen, Voutilainen, Lakka, Jääskeläinen, Poutanen, Auriola, 2018. Analysis by LC-MS/MS of endogenous steroids from human serum, plasma, endometrium and endometriotic tissue. J. Pharm. Biomed. Anal. 152, 165-172.
Jones, Erb, 1968. Levels of 5b-pregnan-3a,6a-diol-20-one in urine and its relationship to other metabolites of progesterone in urine of the nonpregnant domestic sow. J. Anim. Sci. 27, 1054-1058.
Meng, Reyes, Palma, Hernandez, Ribalta, Sjövall, 1997. Profiles of bile acids and progesterone metabolites in the urine and serum of women with intrahepatic cholestasis of pregnancy. J. Hepatol. 27, 346-357.
Moxon, Copley, England, 2010. Quality assurance of canine vaginal cytology: A preliminary study. Theriogenology 74, 479-485.
Niemuller, Shaw, Hodges, 1993. Non-invasive monitoring of ovarian function in Asian elephants (Elephas maximus) by measurement of urinary 5β-Pregnanetriol. J. Reprod. Fertil. 99, 617-625.
O'Connor, Brindle, Holman, Klein, Soules, Campbell, Kohen, Munro, Shofer, Lasley, Wood, 2003. Urinary estrone conjugate and pregnanediol 3-glucuronide enzyme immunoassays for population research. Clin. Chem. 49, 1139-1148.
Senciall, Roberts, Rahal, 1990. A novel chromatographic resolution of 21-hydroxysteroid metabolites of progesterone. J. Steroid Biochem. 35, 151-154.
Stappenbeck, Xiao, Epperson, Riley, Priest, Huang, Nguyen, Jung, Scott Thies, Farouz, 2012. Novel oxysterols activate the Hedgehog pathway and induce osteogenesis. Bioorganic Med. Chem. Lett. 22, 5893-5897.
Suzuki, Murry, Darnel, Sasano, 2002. Progesterone metabolism in human leukemic monoblast U937 cells. Endocr. J.
Wiebe, 2005. Role of progesterone metabolites in mammary cancer. J. Dairy Res. 72, 51-57.

1. (canceled)

2. (canceled)

3. (canceled)

4. (canceled)

5. (canceled)

6. (canceled)

7. Use of a 5α-pregnane-3,6,20-triol isomer or 5α-pregnane-3,6,20-triol glucuronide isomer in determination of progestogen or progestogens in an in vitro urine sample from a non-human animal.

8. The use according to claim 7, characterized in that the 5α-pregnane-3,6,20-triol isomer is selected from the group consisting of 5α-pregnane-3α,6α,20α-triol, 5α-pregnane-3β,6α,20α-triol, 5α-pregnane-3α,6β,20α-triol, 5α-pregnane-3β,6β,20α-triol, 5α-pregnane-3α,6α,20β-triol, 5α-pregnane-3β,6α,20β-triol, 5α-pregnane-3α,6β,20β-triol, and 5α-pregnane-3β,6β,20β-triol.

9. The use according to claim 7, characterized in that the 5α-pregnane-3,6,20-triol glucuronide isomer is selected from the group consisting of 5α-pregnane-3α,6α,20α-triol glucuronide, 5α-pregnane-3β,6α,20α-triol glucuronide, 5α-pregnane-3α,6β,20α-triol glucuronide, 5α-pregnane-3β,6β,20α-triol glucuronide, 5α-pregnane-3α,6α,20β-triol glucuronide, 5α-pregnane-3β,6α,20β-triol glucuronide, 5α-pregnane-3α,6β,20β-triol glucuronide, and 5α-pregnane-3β,6β,20β-triol glucuronide.

10. The use according to claim 7, characterized in that the non-human animal is selected from the group of canines consisting of a domestic dog, wild dogs, wolves, coyote, foxes, bush dog, short-eared dog, jackals, and raccoon dogs.

11. An in vitro method of determining progestogen or progestogens in a urine sample from a non-human animal, characterized in that the method comprises the steps of
providing an in vitro urine sample from a non-human animal,
measuring an amount of 5α-pregnane-3,6,20-triol isomer or 5α-pregnane-3,6,20-triol glucuronide isomer in said sample,
determining the presence of luteinized follicular theca and/or granulosa cells based on the amount of 5α-pregnane-3,6,20-triol isomer or 5α-pregnane-3,6,20-triol glucuronide isomer in the sample,
wherein the progestogen level in the sample is indicative by the presence of luteinized follicular theca and/or granulosa cells.

12. The method according to claim 11, characterized in that the animal is selected from the group of canines consisting of a domestic dog, wild dogs, wolves, coyotes, foxes, a bush dog, a short-haired dog, jackals, and raccoon dogs.

13. The method according to claim 11, characterized in that the measurement is carried out using a method selected from the group consisting of a binding assay, an immunoassay, mass spectrometry, nuclear magnetic resonance, and chromatography.

14. (canceled)

15. The method according to claim 11, characterized in that the 5α-pregnane-3,6,20-triol isomer is selected from the group consisting of 5α-pregnane-3α,6α,20α-triol, 5α-pregnane-3β,6α,20α-triol, 5α-pregnane-3α,6β,20α-triol, 5α-pregnane-3β,6β,20α-triol, 5α-pregnane-3α,6α,20β-triol, 5α-pregnane-3β,6α,20β-triol, 5α-pregnane-3α,6β,20β-triol, and 5α-pregnane-3β,6β,20β-triol.

16. The method according to claim 11, characterized in that the 5α-pregnane-3,6,20-triol glucuronide isomer is selected from the group consisting of 5α-pregnane-3α,6α,20α-triol glucuronide, 5α-pregnane-3β,6α,20α-triol glucuronide, 5α-pregnane-3α,6β,20α-triol glucuronide, 5α-pregnane-3β,6β,20α-triol glucuronide, 5α-pregnane-3α,6α,20β-triol glucuronide, 5α-pregnane-3β,6α,20β-triol glucuronide, 5α-pregnane-3α,6β,20β-triol glucuronide, and 5α-pregnane-3β,6β,20β-triol glucuronide.

17. The use according to claim 8, characterized in that the non-human animal is selected from the group of canines consisting of a domestic dog, wild dogs, wolves, coyote, foxes, bush dog, short-eared dog, jackals, and raccoon dogs.

18. The use according to claim 9, characterized in that the non-human animal is selected from the group of canines consisting of a domestic dog, wild dogs, wolves, coyote, foxes, bush dog, short-eared dog, jackals, and raccoon dogs.

19. The method according to claim 15, characterized in that the animal is selected from the group of canines consisting of a domestic dog, wild dogs, wolves, coyotes, foxes, a bush dog, a short-haired dog, jackals, and raccoon dogs.

20. The method according to claim 16, characterized in that the animal is selected from the group of canines consisting of a domestic dog, wild dogs, wolves, coyotes, foxes, a bush dog, a short-haired dog, jackals, and raccoon dogs.

21. The method according to claim 12, characterized in that the measurement is carried out using a method selected from the group consisting of a binding assay, an immunoassay, mass spectrometry, nuclear magnetic resonance, and chromatography.

22. The method according to claim 15, characterized in that the measurement is carried out using a method selected from the group consisting of a binding assay, an immunoassay, mass spectrometry, nuclear magnetic resonance, and chromatography.

23. The method according to claim 16, characterized in that the measurement is carried out using a method selected from the group consisting of a binding assay, an immunoassay, mass spectrometry, nuclear magnetic resonance, and chromatography.
